(19) United States
(12) Patent Application Publication
(10) Pub. No.: US 2026/0076601 A1
(43) Pub. Date: Mar. 19, 2026

(54) BIOMETRIC MEASUREMENT DEVICE AND BIOMETRIC MEASUREMENT SYSTEM

(71) Applicant: Panasonic Intellectual Property Management Co., Ltd., Osaka (JP)

(72) Inventors: TAKAMASA ANDO, Osaka (JP); SHUNSUKE IMAI, Kanagawa (JP)

(21) Appl. No.: 19/401,499

(22) Filed: Nov. 26, 2025

Related U.S. Application Data
(63) Continuation of application No. PCT/JP2024/020272, filed on Jun. 4, 2024.

(30) Foreign Application Priority Data
Jun. 13, 2023 (JP) 2023-096703

(51) Int. Cl. A61B 5/18; A61B 5/00

(52) U.S. Cl. CPC A61B 5/18; A61B 5/0075; A61B 5/0077; A61B 5/6893; A61B 5/725

(57) ABSTRACT

A biometric measurement device includes a bandpass filter, an optical detection device, and a processing circuit, the optical detection device includes a first filter configured to transmit red light, a second filter configured to transmit one of green light and blue light, a first optical detection element detecting light from a living body through the first filter, and a second optical detection element detecting the light through the second filter, the processing circuit generates biological information of the living body based on first light detected by the first optical detection element and second light detected by the second optical detection element, and the bandpass filter suppresses transmission of at least a part of light in a wavelength range greater than or equal to 550 nm and less than or equal to 600 nm included in light from the living body incident on the first optical detection element.

### BACKGROUND

### 1. Technical Field

The present disclosure relates to a biometric measurement device and a biometric measurement system.

### 2. Description of the Related Art

Conventionally, a method for acquiring biological information such as pulse wave information on the basis of reflected light generated by irradiating a living body with light is known. According to this method, in a case where the living body moves, the reflected light contains not only biological information, but also body motion information, and it is therefore not easy to accurately acquire the biological information. In view of this, a method for removing body motion information has been proposed.

Japanese Unexamined Patent Application Publication No. 2020-157102 (hereinafter referred to as Patent Literature 1) discloses a pulse wave measurement device that measures a pulse wave of a living body by using a camera. Image data of the living body acquired by the camera contains a green signal that is relatively large in an amount of temporal change caused by a pulse and a red signal that is relatively small in the amount of temporal change. The green signal contains not only pulse information, but also body motion information, whereas the red signal contains only a small amount of pulse information and contains a large amount of body motion information. By correcting the green signal by using the red signal, the body motion information can be removed from the information contained in the green signal, and the pulse information can be thus acquired.

Japanese Unexamined Patent Application Publication No. 2004-261366 (hereinafter referred to as Patent Literature 2) discloses a pulse wave sensor that is used while being attached to a living body. The pulse wave sensor detects reflected light generated by irradiating a living body with green light and outputs a green signal, and detects reflected light generated by irradiating the living body with infrared light and outputs an infrared signal. The green signal contains not only pulse information, but also body motion information, whereas the infrared signal contains only a small amount of pulse information and contains a large amount of body motion information. By comparing a frequency component of the green signal and a frequency component of the infrared signal, the body motion information can be removed from the information contained in the green signal, and the pulse information can be thus acquired.

### SUMMARY

One non-limiting and exemplary embodiment provides a biometric measurement device that can effectively acquire biological information.

In one general aspect, the techniques disclosed here feature a biometric measurement device including: a bandpass filter; an optical detection device; and a processing circuit, in which the optical detection device includes a first filter configured to transmit red light, a second filter configured to transmit one of green light and blue light, a first optical detection element that detects light from a living body through the first filter, and a second optical detection element that detects light from the living body through the second filter, the processing circuit generates biological information of the living body on the basis of first light detected by the first optical detection element and second light detected by the second optical detection element, and the bandpass filter suppresses transmission of at least a part of light in a wavelength range greater than or equal to 550 nm and less than or equal to 600 nm that is included in light from the living body incident on the first optical detection element.

According to the present disclosure, it is possible to provide a biometric measurement device that can effectively acquire biological information.

It should be noted that general or specific aspects of the present disclosure may be implemented as a system, an apparatus, a method, an integrated circuit, a computer program, a computer-readable storage medium such as a storage disc, or any selective combination thereof. Examples of the computer-readable storage medium may include a non-volatile storage medium such as a compact disc-read only memory (CD-ROM). The apparatus may include one or more apparatuses. In a case where the apparatus includes two or more apparatuses, the two or more apparatuses may be disposed in one piece of equipment or may be separately disposed in two or more separate pieces of equipment. In the specification and claims, the “apparatus” can mean not only a single apparatus, but also a system including apparatuses.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a graph illustrating an absorption spectrum of oxygenated hemoglobin in blood;

FIG. 2 is a graph illustrating spectral characteristics of a typical color filter;

FIG. 3 schematically illustrates a configuration of a measurement device according to exemplary Embodiment 1 of the present disclosure;

FIG. 4 is a graph illustrating an example of spectral characteristics of a bandpass filter and an absorption spectrum of oxygenated hemoglobin;

FIG. 5 is a graph illustrating an example of spectral characteristics of a configuration in which a bandpass filter and a typical color filter are combined;

FIG. 6 schematically illustrates an example of time-series data of a first signal and a second signal and an example of biological information;

FIG. 7 schematically illustrates an example of arrangement of color filters in an optical detection device;

FIG. 8 schematically illustrates a configuration of a measurement device according to exemplary Embodiment 2 of the present disclosure;

FIG. 9A schematically illustrates Example 1 of a configuration of a light source;

FIG. 9B is a graph illustrating an example of an emission spectrum of light emitted from the light source;

FIG. 10A schematically illustrates Example 2 of the configuration of the light source;

FIG. 10B is a graph illustrating another example of the emission spectrum of the light emitted from the light source;

FIG. 10C is a graph illustrating still another example of the emission spectrum of the light emitted from the light source;

FIG. 11 schematically illustrates a configuration of a measurement device according to exemplary Embodiment 3 of the present disclosure;

FIG. 12A is a graph illustrating an example of spectral characteristics of a first filter;

FIG. 12B is a graph illustrating an example of spectral characteristics of a second filter;

FIG. 12C is a graph illustrating an example of spectral characteristics of a third filter;

FIG. 13A schematically illustrates another example 1 of spectral characteristics of the bandpass filter;

FIG. 13B schematically illustrates another example 2 of spectral characteristics of the bandpass filter;

FIG. 13C schematically illustrates another example 3 of spectral characteristics of the bandpass filter;

FIG. 14A schematically illustrates still another example 1 of spectral characteristics of the bandpass filter;

FIG. 14B schematically illustrates still another example 2 of spectral characteristics of the bandpass filter;

FIG. 14C schematically illustrates still another example 3 of spectral characteristics of the bandpass filter;

FIG. 15 schematically illustrates a modification of a timing of light emission from the light source;

FIG. 16 schematically illustrates how light from a living body that has passed through a telecentric lens passes through a bandpass filter and enters an optical detection device; and

FIG. 17 schematically illustrates a configuration of a biometric measurement system according to an exemplary embodiment of the present disclosure.

### DETAILED DESCRIPTIONS

In the present disclosure, all or a part of any of circuit, unit, device, part or portion, or any of functional blocks in the block diagrams may be implemented as one or more of electronic circuits including, but not limited to, a semiconductor device, a semiconductor integrated circuit (IC) or a large scale integration (LSI). The LSI or IC can be integrated into one chip, or also can be a combination of plural chips. For example, functional blocks other than a memory may be integrated into one chip. The name used here is LSI or IC, but it may also be called system LSI, VLSI (very large scale integration), or ULSI (ultra large scale integration) depending on the degree of integration. A Field Programmable Gate Array (FPGA) that can be programmed after manufacturing an LSI or a reconfigurable logic device that allows reconfiguration of the connection or setup of circuit cells inside the LSI can be used for the same purpose.

Further, it is also possible that all or a part of the functions or operations of the circuit, unit, device, part or portion are implemented by executing software. In such a case, the software is recorded on one or more non-transitory recording media such as a ROM, an optical disk or a hard disk drive, and when the software is executed by a processor, the software causes the processor together with peripheral devices to execute the functions specified in the software. A system or apparatus may include such one or more non-transitory recording media on which the software is recorded and a processor together with necessary hardware devices such as an interface.

In the present disclosure, “light” means electromagnetic waves including not only visible light (having a wavelength ranging from approximately 360 nm to approximately 800 nm), but also ultraviolet light (having a wavelength ranging from approximately 10 nm to approximately 360 nm) and infrared light (having a wavelength ranging from approximately 800 nm to approximately 1 mm).

Exemplary embodiments of the present disclosure are described below. Each of the embodiments described below illustrates a general or specific example. Numerical values, shapes, materials, constituent elements, the way in which the constituent elements are disposed and connected, steps, the order of steps, and the like illustrated in the embodiments below are examples and do not limit the present disclosure. Among constituent elements in the embodiments below, constituent elements that are not described in independent claims indicating highest concepts are described as optional constituent elements. Each drawing is a schematic view and is not necessarily strict illustration. In the drawings, substantially identical constituent elements are given identical reference signs, and repeated description thereof may be omitted or simplified.

First, before description of the embodiments of the present disclosure, a principle for acquiring biological information on the basis of reflected light generated by irradiating a living body with light is described. In the following description, pulse information is taken as an example of the biological information.

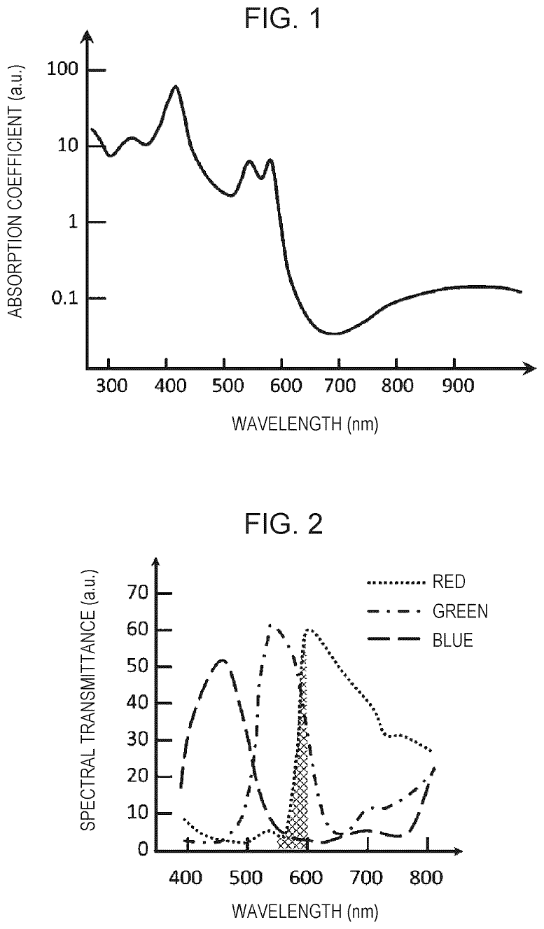

FIG. 1 is a graph illustrating an absorption spectrum of oxygenated hemoglobin in blood. As illustrated in FIG. 1, an absorption coefficient of oxygenated hemoglobin is relatively high in a wavelength range greater than or equal to 300 nm and less than or equal to 600 nm and is relatively low in the other wavelength range in a wavelength range greater than or equal to 300 nm and less than or equal to 900 nm. The absorption coefficient of oxygenated hemoglobin is highest in the vicinity of 420 nm and is relatively high in a wavelength range greater than or equal to 550 nm and less than or equal to 600 nm. Since a degree of absorption of light by oxygenated hemoglobin changes depending on blood flow fluctuations, an intensity of reflected light generated by irradiating a living body with light reflects pulse information.

According to the absorption spectrum of oxygenated hemoglobin, in a case where a living body is irradiated with blue or green light and reflected light thus generated is detected, a blue or green detected signal contains a large amount of pulse information. On the other hand, in a case where a living body is irradiated with red or infrared light and reflected light thus generated is detected, a red or infrared detected signal contains only a small amount of pulse information.

In the present specification, a red wavelength range corresponds to at least a part of a wavelength range greater than or equal to 600 nm and less than or equal to 800 nm. A green wavelength range corresponds to at least a part of a wavelength range greater than or equal to 500 nm and less than or equal to 600 nm. A blue wavelength range corresponds to at least a part of a wavelength range greater than or equal to 360 nm and less than or equal to 500 nm. Light of the red wavelength range is simply referred to as “red light”, light of the green wavelength range is simply referred to as “green light”, and light of the blue wavelength range is simply referred to as “blue light”.

In a case where the living body moves, the blue or green detected signal contains not only pulse information, but also body motion information. On the other hand, the red or infrared detected signal contains only a small amount of pulse information and contains a large amount of body motion information. Therefore, by comparing the blue or green detected signal and the red or infrared detected signal, the body motion information can be removed from the information contained in the blue or green detected signal, and the pulse information can be thus acquired. The red or infrared detected signal is a detected signal for removing the body motion information.

However, in a case where blue, green, and red detected signals are acquired by detecting light from a living body through typical color filters containing pigments or dyes, the following problem can occur. FIG. 2 is a graph illustrating spectral characteristics of typical color filters. In the example illustrated in FIG. 2, the typical color filters include a red filter, a green filter, and a blue filter containing dyes. The dotted line, the line with alternate long and short dashes, and the broken line illustrated in FIG. 2 represent spectral characteristics of the red filter, the green filter, and the blue filter, respectively.

As illustrated in FIG. 2, the blue filter, the green filter, and the red filter each have a broad peak in a corresponding wavelength range. The red filter has relatively high transmittance not only in the red wavelength range, but also in a wavelength range greater than or equal to 550 nm and less than or equal to 600 nm, as indicated by the hatching. Accordingly, in a case where light from a living body is detected through a typical red filter, a red detected signal can contain not only body motion information, but also a certain amount of pulse information. For this reason, even when the blue or green detected signal and the red detected signal are compared, not only body motion information, but also a part of pulse information are removed from the information contained in the blue or green detected signal, and therefore there is a possibility that pulse information cannot be accurately acquired.

In the device of Patent Literature 1, a typical red filter is presumed to be used in the camera. Therefore, in the device of Patent Literature 1, a red detected signal can contain not only body motion information, but also a certain amount of pulse information, and therefore there is a possibility that pulse information cannot be accurately acquired.

In the pulse sensor of Patent Literature 2, reflected light is detected without a color filter. In a case where the pulse sensor is used in a non-contact manner without attachment, a living body is irradiated even with environmental light such as light from indoor lighting and solar light, and therefore other light having a high absorption coefficient may be detected when infrared light from the living body is detected. Therefore, in the pulse sensor of Patent Literature 2, an infrared detected signal may contain not only body motion information, but also a certain amount of pulse information, and therefore there is a possibility that pulse information cannot be accurately acquired.

The inventors of the present invention found the above problem, and arrives at a biometric measurement device according to an embodiment of the present disclosure that can solve the above problem. The biometric measurement device according to the present embodiment is configured so that at least a part of light from a living body in a wavelength range greater than or equal to 550 nm and less than or equal to 600 nm is hard to detect. This can reduce a possibility that biological information such as pulse information is mixed in a detected signal for removing body motion information. As a result, by comparing a detected signal containing not only biological information, but also body motion information and the detected signal for removing body motion information, the biological information can be effectively acquired. The biometric measurement device according to an embodiment of the present disclosure is described below.

### Embodiment 1

### Biometric Measurement Device

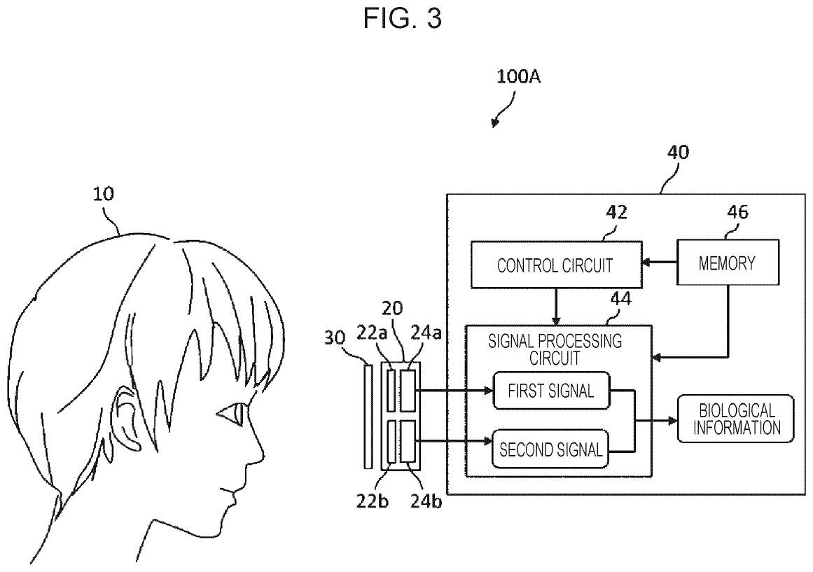

An example of a configuration of a biometric measurement device according to Embodiment 1 of the present disclosure is described below with reference to FIG. 3. In the following description, the biometric measurement device is also referred to simply as a “measurement device”. FIG. 3 schematically illustrates a configuration of the measurement device according to exemplary Embodiment 1 of the present disclosure. A measurement device 100A illustrated in FIG. 3 acquires biological information of a living body 10 in a non-contact manner by detecting light from the living body 10. The light from the living body 10 is, for example, generated by irradiation of the living body 10 with environmental light. Although a person is illustrated as an example of the living body 10, the living body 10 is not limited to this example. The living body 10 may be, for example, an animal. An irradiated portion of the living body 10 can be, for example, a face, an arm, a hand, or a finger of the living body 10. The living body 10 may be stationary or may be in motion.

As illustrated in FIG. 3, the measurement device 100A includes an optical detection device 20, a bandpass filter 30, and a processing device 40. The optical detection device 20 includes a first filter 22a configured to transmit red light to and a second filter 22b configured to transmit one of green light and blue light. The optical detection device 20 further includes a first optical detection element 24a that detects light from the living body 10 through the first filter 22a and a second optical detection element 24b that detects light from the living body 10 through the second filter 22b. The first optical detection element 24a outputs a first signal based on first light detected by the first optical detection element 24a. The second optical detection element 24b outputs a second signal based on second light detected by the second optical detection element 24b. The second optical detection element 24b detects light that has passed through the second filter 22b and the bandpass filter 30 and has at least a wavelength less than or equal to 550 nm. The first signal is a signal corresponding to an intensity of the first light, and the second signal is a signal corresponding to an intensity of the second light.

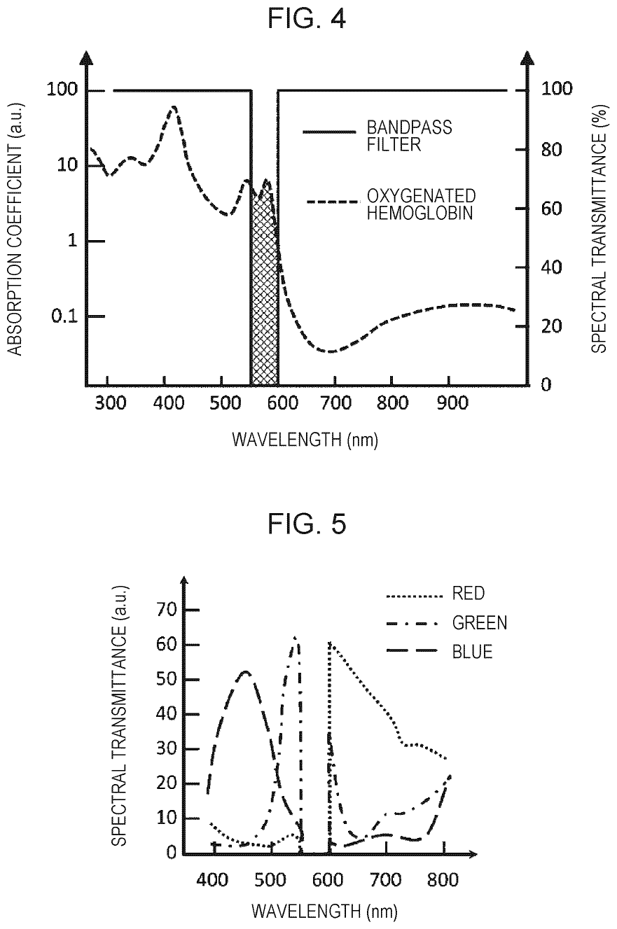

The bandpass filter 30 suppresses transmission of at least part of light in a wavelength range greater than or equal to 550 nm and less than or equal to 600 nm that is included in light from the living body 10 that is incident on the first filter 22a. FIG. 4 is a graph illustrating an example of spectral characteristics of the bandpass filter 30 and an absorption spectrum of oxygenated hemoglobin. The solid line illustrated in FIG. 4 represents the spectral characteristics of the bandpass filter 30, and the broken line illustrated in FIG. 4 represents the absorption spectrum of oxygenated hemoglobin. In the example illustrated in FIG. 4, the bandpass filter 30 has almost zero spectral transmittance in the wavelength range greater than or equal to 550 nm and less than or equal to 600 nm, and has almost 100% spectral transmittance in the other wavelength ranges. It is therefore possible to effectively suppress transmission of light from the living body 10 in the wavelength range greater than or equal to 550 nm and less than or equal to 600 nm. The light from the living body 10 that has passed through the bandpass filter 30 does not reflect the hatched part of the absorption spectrum of oxygenated hemoglobin.

FIG. 5 is a graph illustrating an example of spectral characteristics of a configuration combining the bandpass filter 30 and typical color filters. As illustrated in FIG. 5, in the configuration combining the bandpass filter 30 and typical color filters, the spectral transmittance is almost zero in the wavelength range greater than or equal to 550 nm and less than or equal to 600 nm. Therefore, in a case where the living body 10 moves, the second signal contains not only biological information, but also body motion information, whereas the first signal contains almost no biological information and contains a large amount of body motion information. The bandpass filter 30 can thus effectively suppress mixture of biological information in the first signal.

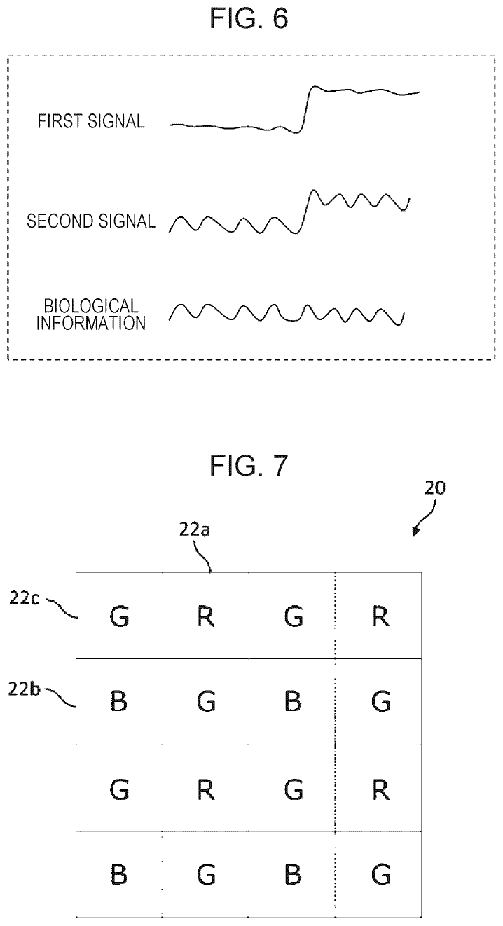

FIG. 6 schematically illustrates an example of time-series data of the first signal and the second signal and an example of biological information. The vertical direction of FIG. 6 represents a signal intensity, and the horizontal direction of FIG. 6 represents time. Pulse information is illustrated as the biological information illustrated in FIG. 6. In a case where the living body 10 moves during measurement, the time-series data of the second signal fluctuates continuously reflecting the pulse information and shifts markedly reflecting body motion information at a certain timing. On the other hand, the time-series data of the first signal shifts markedly reflecting body motion information at a certain timing, but does not reflect the pulse information due to the bandpass filter 30 and therefore hardly fluctuates. As a result, the processing device 40 can remove body motion information from the information contained in the second signal by comparing the first signal and the second signal while suppressing removal of a part of the pulse information from the information contained in the second signal, and therefore can more accurately acquire the pulse information.

Therefore, the measurement device 100A according to Embodiment 1 can more accurately acquire biological information of the living body 10 than a configuration obtained by removing the bandpass filter 30 from the measurement device 100A. This leads to effective acquisition of the biological information of the living body 10.

Configurations of the optical detection device 20, the bandpass filter 30, and the processing device 40 are described in detail below.

### Optical Detection Device 20

As illustrated in FIG. 3, the optical detection device 20 includes at least one first filter 22a and at least one second filter 22b. The first filter 22a and the second filter 22b have been described above. The optical detection device 20 may further include at least one third filter configured to transmit the other one of the green light and the blue light. The number of first filters 22a may be one or may be more than one. The same applies to the number of second filters 22b and the number of third filters.

The first filter 22a may be, for example, designed to transmit light having a wavelength of 690 nm with high spectral transmittance for the following reason. The high spectral transmittance can be, for example, greater than or equal to 60%, greater than or equal to 80%, greater than or equal to 90%, or greater than or equal to 95%. As illustrated in FIG. 4, the absorption coefficient of oxygenated hemoglobin is minimum at the wavelength of 690 nm in a wavelength range greater than or equal to 300 nm and less than or equal to 900 nm, and therefore light from the living body 10 that has a wavelength of 690 nm contains only a small amount of biological information. Therefore, by thus designing the first filter 22a, an amount of biological information contained in the first signal can be effectively reduced.

The typical color filters described above can be used as the first filter 22a, the second filter 22b, and the third filter. Such color filters are widely available, low-cost, and suitable for mass production.

As illustrated in FIG. 2, spectral characteristics of the typical red filter have a broad peak in a wavelength range greater than or equal to 550 nm and less than or equal to 800 nm. A peak wavelength of the spectral characteristics of the typical red filter is within the wavelength range greater than or equal to 550 nm and less than or equal to 800 nm. Spectral transmittance at the peak wavelength can be maximum spectral transmittance in the wavelength range of visible light. The spectral characteristics of the typical red filter has spectral transmittance greater than or equal to 30% in at least a part of the wavelength range greater than or equal to 550 nm and less than or equal to 600 nm. The spectral characteristics of the typical red filter further has spectral transmittance less than or equal to 20% in a wavelength range less than or equal to 500 nm in the wavelength range of visible light.

Spectral characteristics of the typical green filter has a broad peak in a wavelength range greater than or equal to 450 nm and less than or equal to 650 nm. A peak wavelength of the spectral characteristics of the typical green filter is within the wavelength range greater than or equal to 450 nm and less than or equal to 650 nm. Spectral characteristics of the typical blue filter has a broad peak in a wavelength range greater than or equal to 360 nm and less than or equal to 550 nm. A peak wavelength of the spectral characteristics of the typical blue filter is within the wavelength range greater than or equal to 360 nm and less than or equal to 550 nm.

The optical detection device 20 includes at least one first optical detection element 24a and at least one second optical detection element 24b. The first optical detection element 24a and the second optical detection element 24b have been described above. The optical detection device 20 may further include at least one third optical detection element that detects light from the living body 10 through the third filter. The third optical detection element outputs a third signal based on third light detected by the third optical detection element. The third signal is a signal corresponding to an intensity of the third light.

In a case where the first filter 22a and the first optical detection element 24a correspond on a one-to-one basis, the number of first optical detection elements 24a is equal to the number of first filters 22a. Similarly, the number of second optical detection elements 24b is equal to the number of second filters 22b. The number of third optical detection elements is equal to the number of third filters.

Alternatively, in a case where the first filter 22a overlaps two or more first optical detection elements 24a when viewed in a direction in which light from the living body 10 is incident, the number of first optical detection elements 24a may be larger than the number of first filters 22a. Similarly, the number of second optical detection elements 24b may be larger than the number of second filters 22b. The number of third optical detection elements may be larger than the number of third filters.

The optical detection device 20 can be, for example, a color camera including typical color filters and an image sensor. In this case, the optical detection device 20 can acquire a color image of the living body 10. The image sensor includes optical detection elements arranged two-dimensionally. The image sensor can be a charge-coupled device (CCD) sensor or a complementary metal oxide semiconductor (CMOS) sensor. Each of the color filters may be directly provided on a corresponding one of the optical detection elements or may be indirectly provided with another member interposed therebetween, for example.

FIG. 7 schematically illustrates an example of arrangement of the color filters in the optical detection device 20. “R”, “G”, and “B” illustrated in FIG. 7 represent a red filter, a green filter, and a blue filter, respectively. In the example illustrated in FIG. 7, the first filter 22a is a red filter, the second filter 22b is a blue filter, and the third filter 22c is a green filter. In the example illustrated in FIG. 7, the arrangement of the color filters is Bayer arrangement. The Bayer arrangement has units arranged two-dimensionally. The units include filters in two rows and two columns. In each of the units, two green filters are arranged as diagonal elements, and a red filter and a blue filter are arranged as non-diagonal elements. Alternatively, in each of the units, two red filters may be arranged as diagonal elements, and a green filter and a blue filter may be arranged as non-diagonal elements. In each of the units, two blue filters may be arranged as diagonal elements, and a green filter and a red filter may be arranged as non-diagonal elements. The arrangement of the color filters is not limited to Bayer arrangement, and may be stripe arrangement, delta arrangement, mosaic arrangement, or Pentile arrangement. In the optical detection device 20, an infrared filter may be used instead of the red filter.

### Bandpass Filter 30

The bandpass filter 30 suppresses transmission of at least a part of light in the wavelength range greater than or equal to 550 nm and less than or equal to 600 nm that is included in light from the living body 10 that is incident on the first filter 22a. In a case where the typical red filter is used as the first filter 22a, the first filter 22a transmits not only red light, but also light in the wavelength range greater than or equal to 550 nm and less than or equal to 600 nm, as illustrated in FIG. 2. Therefore, in the configuration obtained by removing the bandpass filter 30 from the measurement device 100A, there is a possibility that biological information is mixed in the first signal. On the other hand, according to the measurement device 100A, the possibility can be reduced by the bandpass filter 30. The first filter 22a, the bandpass filter 30, and the first optical detection element 24a may be arranged in this order so that light from the living body 10 is incident from the first filter 22a toward the first optical detection element 24a. That is, the bandpass filter 30 may be placed between the first filter 22a and the first optical detection element 24a.

The wavelength range in which light transmission is suppressed by the bandpass filter 30 may be the whole wavelength range greater than or equal to 550 nm and less than or equal to 600 nm or may be a part of the wavelength range greater than or equal to 550 nm and less than or equal to 600 nm. The bandpass filter 30 may, for example, suppress transmission of light in a wavelength range greater than or equal to 570 nm and less than or equal to 600 nm and allow transmission of light in the other wavelength range in the wavelength range greater than or equal to 550 nm and less than or equal to 600 nm.

The expression “suppress transmission of at least a part of light in the wavelength range greater than or equal to 550 nm and less than or equal to 600 nm” means a case where spectral transmittance in at least a part of the wavelength range greater than or equal to 550 nm and less than or equal to 600 nm is less than or equal to 20%. The spectral transmittance may be less than or equal to 10%, may be less than or equal to 5%, or may be less than or equal to 1%. As the spectral transmittance becomes closer to zero, biological information can be acquired more accurately on the basis of the first signal and the second signal.

In the example illustrated in FIG. 4, the spectral transmittance of the bandpass filter 30 is almost 100% in the remaining part of the wavelength range of visible light other than the wavelength range in which light transmission is suppressed. However, the spectral transmittance of the bandpass filter 30 need not be almost 100% in the remaining wavelength range. The spectral transmittance can be, for example, greater than or equal to 60%, greater than or equal to 80%, greater than or equal to 90%, or greater than or equal to 95%. As the spectral transmittance becomes higher, the first signal can contain a larger amount of body motion information, and the second signal can contain a larger amount of biological information.

The bandpass filter 30 is placed so as to overlap the first filter 22a when viewed in the direction in which light from the living body 10 is incident. On the other hand, the bandpass filter 30 may be placed so as to overlap the second filter 22b and the third filter 22c or may be placed so as not to overlap the second filter 22b and the third filter 22c. However, in a case where the optical detection device 20 is a color camera, not only the first filters 22a, but also the second filters 22b and the third filters 22c are arranged two-dimensionally. It is therefore easier to arrange the bandpass filter 30 in a case where the bandpass filter 30 is placed so as to overlap the whole optical detection device 20. Furthermore, by separately preparing the color filters and the bandpass filter 30, a color camera suitable for mass production can be used as the optical detection device 20, and a production cost of the measurement device 100A can be reduced.

In a case where the bandpass filter 30 overlaps the second filter 22b and the third filter 22c, an amount of at least a part of light in the wavelength range greater than or equal to 550 nm and less than or equal to 600 nm that is incident on the second optical detection element and the third optical detection element decreases. Even in this case, the second signal and the third signal contain a sufficient amount of biological information although an amount of biological information contained in the second signal and the third signal decreases. This is because in the configuration combining the bandpass filter 30 and the green filter, the spectral transmittance is sufficiently high in a wavelength range greater than or equal to 500 nm and less than or equal to 550 nm, as illustrated in FIG. 5. Similarly, this is because in the configuration combining the bandpass filter 30 and the blue filter, the spectral transmittance is sufficiently high in a wavelength range greater than or equal to 400 nm and less than or equal to 500 nm. As illustrated in FIG. 4, the absorption coefficient of oxygenated hemoglobin is sufficiently high in these wavelength ranges.

The bandpass filter 30 can be, for example, an interference filter including a dielectric multi-layer. The interference filter including the dielectric multi-layer can realize a rapid change in spectral characteristics. It is therefore possible to effectively suppress transmission of at least a part of light in the wavelength range greater than or equal to 550 nm and less than or equal to 600 nm in the wavelength range of visible light while effectively transmitting light in the remaining wavelength range.

Alternatively, the bandpass filter 30 may be, for example, a filter containing a pigment or a dye. Such a filter is advantageous in that the filter does not have dependence on an incident angle of incident light, unlike an interference filter including a dielectric multi-layer.

### Processing Device 40

As illustrated in FIG. 3, the processing device 40 includes a control circuit 42, a signal processing circuit 44, and a memory 46. The control circuit 42 controls processing operation of the signal processing circuit 44.

The signal processing circuit 44 generates biological information of the living body 10 on the basis of the first light and the second light, more specifically, on the basis of the first signal and the second signal. A method for generating biological information on the basis of the first signal and the second signal will be described later. The signal processing circuit 44 may increase the intensities of the first signal, the second signal, and the third signal by adjusting an exposure period per frame when acquiring time-series data of the first signal, the second signal, and the third signal.

The control circuit 42 can be, for example, a combination of a processor and a memory or an integrated circuit such as a microcontroller including a processor and a memory. For example, the processor executes a computer program stored in the memory 46, and thus the control circuit 42 causes the signal processing circuit 44 to perform signal processing.

The signal processing circuit 44 can be realized, for example, by a digital signal processor (DSP), a programmable logic device (PLD) such as a field programmable gate array (FPGA), or a combination of a central processing unit (CPU) or a graphics processing unit (GPU) and a computer program. The processor executes a computer program stored in the memory 46, and thus the signal processing circuit 44 performs signal processing.

The control circuit 42 and the signal processing circuit 44 may be a single unified circuit or may be separate individual circuits. At least one of the control circuit 42, the signal processing circuit 44, and the memory 46 may be, for example, a constituent element of an external device such as a server installed in a remote place. In this case, the external device such as a server transmits and receives data to and from a remaining constituent element by wireless communication or wired communication.

In the present specification, the control circuit 42 and the signal processing circuit 44 are also collectively referred to as a “processing circuit”. Operation of the control circuit 42 and the signal processing circuit 44 may be handled as operation of the processing circuit.

### Other Constituent Elements

In addition to the constituent elements described above, the measurement device 100A may further include, for example, an optical system placed before the bandpass filter 30. The optical system can include, for example, a large-aperture lens having a f-number of 1 or less. Such a large-aperture lens can improve sensitivity of light detection of the optical detection device 20.

### Method for Generating Biological Information

A method for generating biological information on the basis of the first signal and the second signal is described below.

### Difference

The signal processing circuit 44 can generate biological information on the basis of a difference between a temporal change of the intensity of the first signal and a temporal change of the intensity of the second signal. More specifically, the signal processing circuit 44 acquires biological information contained in the second signal by subtracting data obtained by multiplying the time-series data of the first signal by an appropriate coefficient from the time-series data of the second signal. The coefficient can be, for example, appropriately determined at a time of calibration before shipment of the measurement device 100A.

According to the method for generating biological information by using the difference, a computational load on the signal processing circuit 44 can be reduced.

### Independent Component Analysis or Principal Component Analysis

The signal processing circuit 44 may apply independent component analysis or principal component analysis to the time-series data of the first signal and the time-series data of the second signal. The independent component analysis or principal component analysis can separate biological information and body motion information from these two pieces of time-series data.

Alternatively, the signal processing circuit 44 may apply independent component analysis or principal component analysis to the time-series data of the third signal in addition to the time-series data of the first signal and the time-series data of the second signal. As a result, biological information, body motion information, and noise information can be separated from these three pieces of time-series data. The noise information can be, for example, information concerning Schottky noise.

According to the method for generating biological information by independent component analysis or principal component analysis, biological information can be more accurately acquired than the method for generating biological information by using the difference.

### Machine Learning

The signal processing circuit 44 may acquire biological information from the time-series data of the first signal and the time-series data of the second signal by machine learning.

The signal processing circuit 44 acquires the first signal and the second signal by the measurement device 100A, and acquires biological information by another measurement device. The signal processing circuit 44 creates a regression model for predicting pulse information from the first signal and the second signal by machine learning while using, as ground truth data, the biological information acquired by the other measurement device. The signal processing circuit 44 acquires biological information from the time-series data of the first signal and the time-series data of the second signal on the basis of the regression model thus created.

As the machine learning, logistic regression or support vector machine may be used or deep learning, which is one kind of machine learning, may be used. Time-series data of biological information may be predicted by using Long Short-Term Memory (LSTM) used for prediction of time-series data.

In a case where the biological information is pulse information, an electrocardiographic sensor can be, for example, used as the other measurement device for acquiring the ground truth data. In a case where a photoplethysmogram is measured at a fingertip by using photoplethysmography, the fingertip is stationary so that the photoplethysmogram does not contain a shift caused by body motion. Since a photoplethysmographic signal is similar to a signal acquired by the measurement device 100A, accuracy of regression can be increased.

According to the method for generating biological information by using machine learning, tendency of superposition of body motion information in the first signal may be different from tendency of superposition of body motion information in the second signal, unlike the example illustrated in FIG. 6. The difference in tendency of superposition can, for example, occur under an environment where an illuminance distribution on the living body 10 differs between the red light for acquiring the first signal and the one of the green light and the blue light for acquiring the second signal. Training data may be collected by acquiring biological information under the environment. By using the training data thus collected, a regression model reflecting the difference in tendency of superposition can be created. Therefore, even in a case where the tendency of superposition of body motion information in the first signal is different from the tendency of superposition of body motion information in the second signal, body motion information can be removed from the information contained in the second signal while suppressing removal of a part of biological information from the information contained in the second signal, and it is therefore possible to more accurately acquire biological information.

### Kind of Biological Information

The biological information can be, for example, pulse information, as described above. Alternatively, the biological information may be blood flow information. A color of an irradiated portion of the living body 10 changes due to changes in blood flow. Blood flow information can be acquired from a temporal change in color. The color can be known on the basis of the first signal, the second signal, and the third signal. In a case where the irradiated portion of the living body 10 is a face, the blood flow information is blood flow information of the face.

Alternatively, the biological information may be information on oxygen saturation. The oxygen saturation can be calculated by first concentration/(first concentration+second concentration) where the first concentration is a concentration of oxygenated hemoglobin in blood and the second concentration is a concentration of deoxygenated hemoglobin in blood. An absorption spectrum of deoxygenated hemoglobin is different from the absorption spectrum of oxygenated hemoglobin illustrated in FIG. 4.

The oxygen saturation can be calculated on the basis of the first signal and the second signal, for example, as follows. By solving a system of equations expressed by the following equations (1) and (2), change amounts of the concentration of oxygenated hemoglobin (HbO₂) and the concentration of deoxygenated hemoglobin (Hb) in blood from initial values are found.

$${ε}_{OXY}^{1} Δ {Hb O}_{2} + {ε}_{deOXY}^{1} Δ Hb = - ln \frac{{I}_{now}^{1}}{{I}_{ini}^{1}} \qquad ( 1 )$$

$${ε}_{OXY}^{2} Δ {Hb O}_{2} + {ε}_{deOXY}^{2} Δ Hb = - ln \frac{{I}_{now}^{2}}{{I}_{ini}^{2}} \qquad ( 2 )$$

ΔHbO₂ and ΔHb represent the change amounts of the concentrations of HbO₂ and Hb in blood from the initial values, respectively. ε¹_{oxy} and ε¹_{deOXY} represent molar adsorption coefficients of HbO₂ and Hb for the red light for acquiring the first signal, respectively. ε²_{OXY} and ε²_{deOXY} represent molar adsorption coefficients of HbO₂ and Hb for the green light or the blue light for acquiring the second signal, respectively. I¹ᵢₙᵢ and I²_{now} represent intensities of the first signal at an initial time and a measurement time, respectively. I²ᵢₙᵢ and I²_{now} represent intensities of the second signal at the initial time and the measurement time, respectively.

The above system of equations can be solved since ε¹_{OXY} and ε¹_{deOXY} differ relatively markedly although ε²_{OXY} and ε²_{deOXY} do not differ markedly. When ΔHbO₂ and ΔHb are found, the oxygen saturation can be calculated from a predetermined equation.

Alternatively, the biological information may be blood pressure information. When pulse information is known, a pulse wave propagation time can be found from a waveform of a pulse wave. A systolic blood pressure P is found from a relationship P=a×lnΔT+b where P is the systolic blood pressure and ΔT is the pulse wave propagation time. The coefficient a and the coefficient b can be, for example, appropriately determined at a time of calibration before shipment of the measurement device 100A.

### Face Tracking

In a case where the optical detection device 20 is a color camera, the face of the living body 10 can be tracked as follows. RGB (Red, Green, Blue) color spaces obtained by the first signal, the second signal, and the third signal are converted into color spaces that include hue. The color spaces that include hue can be, for example, an HSV (Hue, Saturation, Value) color space, an HSL (Hue, Saturation, Luminance) color space, and an HSI (Hue, Saturation, Intensity) color space.

The signal processing circuit 44 generates hue information of the living body 10 on the basis of the third light in addition to the first light and the second light, more specifically, the third signal in addition to the first signal and the second signal. The signal processing circuit 44 tracks the face of the living body 10 on the basis of the hue information. The expression “tracking the face” means specifying a position of the face, more specifically, specifying an optical detection element corresponding to the position of the face among optical detection elements included in the color camera. The expression “on the basis of the hue information” means matching against a preset hue condition, more specifically, setting a condition that it is determined that a region is human skin in a case where the hue is within a predetermined range and checking whether the acquired hue matches the condition.

A more specific configuration of the optical detection device 20 and more specific operation of the signal processing circuit 44 are as follows.

The optical detection device 20 includes the first filters 22a, the second filters 22b, and the third filters. The first filters 22a have an identical configuration. The same applies to the second filters 22b and the third filters. The optical detection device 20 further includes the first optical detection elements 24a, the second optical detection elements 24b, and the third optical detection elements. The first optical detection elements 24a have an identical configuration. The same applies to the second optical detection elements 24b and the third optical detection elements.

The signal processing circuit 44 generates hue information of the living body 10 on the basis of light detected by each of the first optical detection elements 24a, light detected by each of the second optical detection elements 24b, and light detected by each of the third optical detection elements. The signal processing circuit 44 determines a first optical detection element 24a, a second optical detection element 24b, and a third optical detection element that correspond to the position of the face among the first optical detection elements 24a, the second optical detection elements 24b, and the third optical detection elements included in the optical detection device 20 on the basis of the hue information.

Therefore, even in a case where a person moves during measurement, it can be determined in which portion of an image acquired by the color camera the face is present. The first signal and the second signal can be acquired from the portion of the image where the face is present, and biological information can be acquired on the basis of the first signal and the second signal thus acquired. Furthermore, influence of partial overlapping between the spectral characteristics of the red filter and the spectral characteristics of the one of the green filter and the blue filter can be reduced by the bandpass filter 30, and therefore accuracy of the hue information can be improved. The portion to be tracked is not limited to a face, and skin may be tracked, that is, a position of skin may be specified.

Therefore, according to the measurement device 100A according to Embodiment 1, the second signal contains not only biological information, but also body motion information, whereas the first signal contains a large amount of body motion information and contains only a small amount of biological information due to the bandpass filter 30. As a result, biological information of the living body 10 can be effectively acquired on the basis of the first signal and the second signal.

### Embodiment 2

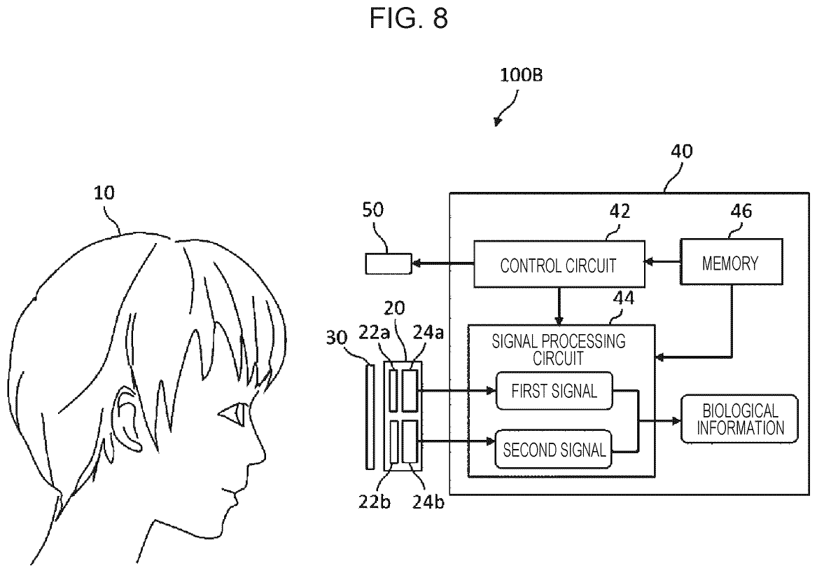

An example of a configuration of a measurement device according to Embodiment 2 of the present disclosure is described below with reference to FIG. 8. FIG. 8 schematically illustrates the configuration of the measurement device according to the exemplary Embodiment 2 of the present disclosure. A measurement device 100B illustrated in FIG. 8 further includes a light source 50 that emits light for irradiating a living body 10, unlike the measurement device 100A illustrated in FIG. 3. A control circuit 42 causes the light source 50 to constantly or intermittently emit light for irradiating the living body 10. The light source 50 makes it possible to acquire biological information of the living body 10 without environmental light.

The light source 50 emits red light and one of green light and blue light for irradiating the living body 10. The light source 50 may further emit the other one of the green light and blue light for irradiating the living body 10. The red light is useful for acquisition of a first signal, and the one of the green light and the blue light is useful for acquisition of a second signal. The other one of the green light and the blue light is useful for acquisition of a third signal.

Note that the measurement device 100B may further include a diffusion panel that diffuses light emitted from the light source 50. By uniformly irradiating the living body 10 with the diffused light, biological information can be evaluated on the basis of the same criterion in any regions of an irradiated portion of the living body 10. These regions of the irradiated portion of the living body 10 can be, for example, left and right regions of a forehead.

### Example 1 of Configuration of Light Source 50

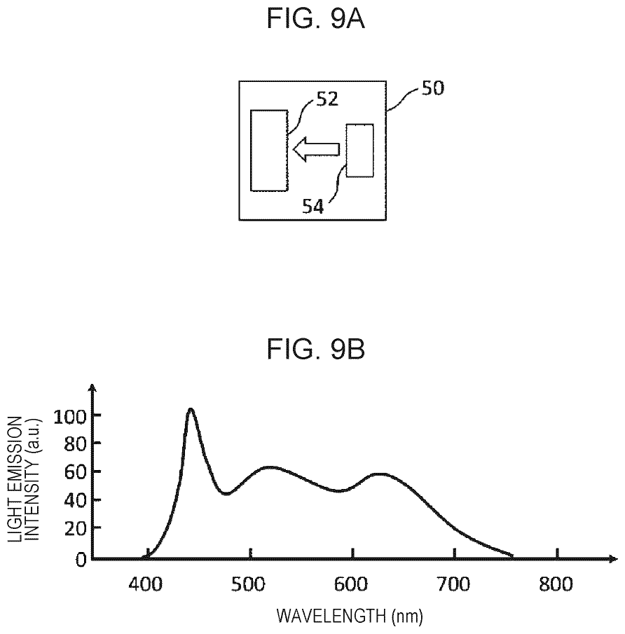

FIG. 9A schematically illustrates Example 1 of the configuration of the light source 50. As illustrated in FIG. 9A, the light source 50 includes, for example, a wavelength conversion element 52 and a light-emitting element 54 that emits excitation light for exciting the wavelength conversion element 52. The white arrow illustrated in FIG. 9A represents the excitation light. The light-emitting element 54 can be, for example, a laser diode (LD) or a light-emitting diode (LED).

In a case where the light-emitting element 54 emits blue light, the wavelength conversion element 52 can include, for example, a fluorescent body that absorbs the blue light and emits yellow light. The yellow light includes red light and green light. The red light included in the yellow light is useful for acquisition of the first signal. The blue light is useful for acquisition of one of the second signal and the third signal, and the green light included in the yellow light is useful for acquisition of the other one of the second signal and the third signal.

A part of the blue light emitted from the light-emitting element 54 is converted into yellow light by the fluorescent body, and a remaining part passes through the wavelength conversion element 52 without being converted. As a result, the light source 50 emits white light in which the yellow light and the blue light are mixed. An average color rendering index Ra of the light source 50 can be, for example, greater than or equal to 80. As the average color rendering index Ra becomes closer to 100, a perceived color of an object irradiated with light emitted from the light source 50 becomes closer to a perceived color of the object irradiated with solar light.

Since environmental light such as light from indoor lighting and solar light is white light, the white light emitted from the light source 50 can lessen a psychological burden on the living body 10 during measurement. Furthermore, as the average color rendering index Ra becomes closer to 100, the white light contains more light having a wavelength of 690 nm. Therefore, in a case where the first filter 22a is designed to allow transmission of light having a wavelength of 690 nm with high spectral transmittance as described above, an SN ratio of the first signal can be improved.

Alternatively, in a case where the light-emitting element 54 emits blue light, the wavelength conversion element 52 can include, for example, a first fluorescent body and a second fluorescent body described below. The first fluorescent body absorbs the blue light and emits red light. The second fluorescent body absorbs the blue light and emits green light.

A part of the blue light emitted from the light-emitting element 54 is converted into red light by the first fluorescent body, another part of the blue light emitted from the light-emitting element 54 is converted into green light by the second fluorescent body, and a remaining part passes through the wavelength conversion element 52 without being converted. As a result, the light source 50 emits white light in which the red light, the green light, and the blue light are mixed. The average color rendering index Ra of this light source 50 can be, for example, greater than or equal to 90.

FIG. 9B is a graph illustrating an example of an emission spectrum of light emitted from the light source 50. In the example illustrated in FIG. 9B, the light-emitting element 54 emits blue light, and the wavelength conversion element 52 includes the first fluorescent body and the second fluorescent body. As illustrated in FIG. 9B, the emission spectrum has a largest peak in a blue wavelength range. The emission spectrum further has a peak in a green wavelength range and has a peak in a red wavelength range. Since the emission spectrum has a peak in each of the wavelength ranges of the three colors, the average color rendering index Ra is high. Note that in a case where it is unnecessary to acquire the third signal, the wavelength conversion element 52 need not necessarily include the second fluorescent body that absorbs the blue light and emits the green light.

Alternatively, in a case where the light-emitting element 54 emits ultraviolet light, the wavelength conversion element 52 can include, for example, a first fluorescent body, a second fluorescent body, and a third fluorescent body described below. The first fluorescent body absorbs the ultraviolet light and emits red light. The second fluorescent body absorbs the ultraviolet light and emits one of green light and blue light. The third fluorescent body absorbs the ultraviolet light and emits the other one of the green light and the blue light.

A part of the ultraviolet light emitted from the light-emitting element 54 is converted into red light by the first fluorescent body, another part of the ultraviolet light emitted from the light-emitting element 54 is converted into one of green light and blue light by the second fluorescent body, and still another part of the ultraviolet light emitted from the light-emitting element 54 is converted into the other one of the green light and the blue light by the third fluorescent body. In a case where the wavelength conversion element 52 includes the first fluorescent body, the second fluorescent body, and the third fluorescent body, the light source 50 emits white light in which the red light, the green light, and the blue light are mixed. The average color rendering index Ra of this light source 50 can be, for example, greater than or equal to 95. Note that in a case where it is unnecessary to acquire the third signal, the wavelength conversion element 52 need not necessarily include the third fluorescent body that absorbs the ultraviolet light and emits the other one of the green light and the blue light.

As illustrated in FIG. 9A, the light source 50 including the wavelength conversion element 52 can emit red light and one of green light and blue light from the wavelength conversion element 52, that is, from the same position. Since the living body 10 is irradiated with the red light and the one of the green light and the blue light emitted from the same position, body motion information contained in the second signal acquired by the irradiation is completely synchronized with body motion information contained in the first signal acquired by the irradiation. It is therefore possible to more effectively acquire biological information of the living body 10 on the basis of the first signal and the second signal.

### Example 2 of Configuration of Light Source 50

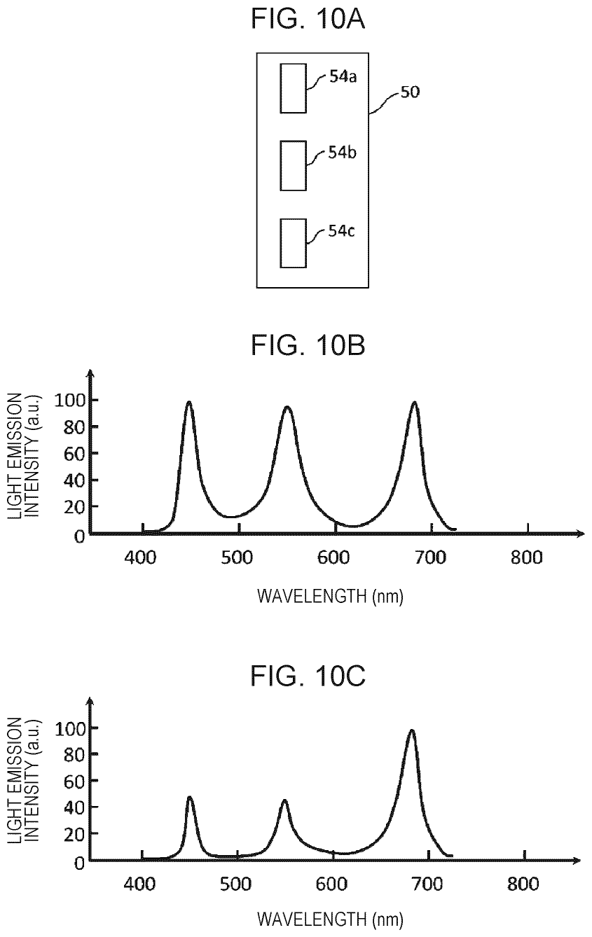

FIG. 10A schematically illustrates Example 2 of the configuration of the light source 50. As illustrated in FIG. 10A, the light source 50 can include, for example, a first light-emitting element 54a, a second light-emitting element 54b, and a third light-emitting element 54c described below. The first light-emitting element 54a emits red light. The second light-emitting element 54b emits one of green light and blue light. The third light-emitting element 54c emits the other one of the green light and the blue light. The first light-emitting element 54a, the second light-emitting element 54b, and the third light-emitting element 54c can be, for example, LDs or LEDs. Note that in a case where it is unnecessary to acquire the third signal, the light source 50 need not necessarily include the third light-emitting element 54c.

The first light-emitting element 54a, the second light-emitting element 54b, and the third light-emitting element 54c can be, for example, placed so that a center-to-center distance between any two of the light-emitting elements is less than or equal to 10 mm. In a case where a distance from the light source 50 to the living body 10 is 30 cm, and the center-to-center distance between any two of the light-emitting elements is larger than 10 mm, an error in signal intensity is larger than 1% due to a difference in illuminance caused by incident angles of light emitted from the two light-emitting elements and entering the living body 10. This error in signal intensity is similar to or greater than a change amount of a signal intensity caused by a pulse. In a case where the center-to-center distance between any two of the light-emitting elements is less than or equal to 10 mm, the error in signal intensity can be reduced.

FIG. 10 is a graph illustrating another example of an emission spectrum of light emitted from the light source 50. In the example illustrated in FIG. 10B, the light source 50 includes the first light-emitting element 54a, the second light-emitting element 54b, and the third light-emitting element 54c. These light-emitting elements are LEDs that are less expensive than LDs.

In a case where all of the light-emitting elements are LEDs, an emission spectrum of light of each color is relatively narrow, and therefore light of a wavelength unnecessary for acquisition of biological information can be reduced as compared with a case where the wavelength conversion element 52 is used as illustrated in FIG. 9A. This can reduce glare. Although an emission spectrum of red light overlaps an emission spectrum of green light in a wavelength range greater than or equal to 550 nm and less than or equal to 600 nm, influence of the overlapping can be reduced by the bandpass filter 30. Note that the emission spectrum such as the one illustrated in FIG. 10B can also be realized by the light source 50 that emits red light, green light, and blue light by exciting three fluorescent bodies by ultraviolet light.

In a case where all of the light-emitting elements are LDs, an emission spectrum of each light is sufficiently narrow, and therefore light of a wavelength unnecessary for acquisition of biological information can be further reduced as compared with the case where all of the light-emitting elements are LEDs. This can further reduce glare.

However, in a case where LDs are used, the light-emitting elements are designed so that an intensity of laser light satisfies class 1 in accordance with laser device safety standards “JIS C 6802” for eye safety.

In a case where LDs are used, an emission spectrum of red light does not overlap an emission spectrum of green light in a wavelength range greater than or equal to 550 nm and less than or equal to 600 nm, but in a case where the measurement device 100A is used in a non-contact manner, light from the living body 10 can include light in the wavelength range greater than or equal to 550 nm and less than or equal to 600 nm due to environmental light. Even in this case, mixture of at least a part of the light in the wavelength range originating from environmental light in the first signal can be suppressed due to the bandpass filter 30.

In a case where LDs or LEDs are used, a peak intensity of the red light, a peak intensity of the green light, and a peak intensity of the blue light need not be the same. FIG. 10C is a graph illustrating still another example of an emission spectrum of light emitted from the light source 50. In the example illustrated in FIG. 10C, the light source 50 includes the first light-emitting element 54a, the second light-emitting element 54b, and the third light-emitting element 54c. These light-emitting elements are LEDs. As illustrated in FIG. 10C, a peak intensity of each of the green light and the blue light is lower than a peak intensity of the red light.

In a case where the peak intensity of the red light, the peak intensity of the green light, and the peak intensity of the blue light are the same as illustrated in FIG. 10B, the following problem can occur. Since the bandpass filter 30 can suppress transmission of at least a part of light in the wavelength range greater than or equal to 550 nm and less than or equal to 600 nm, there is a possibility that an intensity of the first signal becomes lower than an intensity of the second signal and is lower than an intensity of the third signal. Although the intensity of the first signal can be increased by prolonging an exposure period, there is a possibility that the intensity of the second signal and the intensity of the third signal are saturated.

On the other hand, according to the relationship among the peak intensity of the red light, the peak intensity of the green light, and the peak intensity of the blue light illustrated in FIG. 10C, the intensity of the first signal can be increased by prolonging the exposure period, and the possibility that the intensity of the second signal and the intensity of the third signal are saturated can be reduced. In a case where the peak intensity of each of the green light and the blue light is less than or equal to a half of the peak intensity of the red light, the possibility can be effectively reduced.

The intensities of the light emitted from the first light-emitting element 54a, the second light-emitting element 54b, and the third light-emitting element 54c may be adjusted so that the peak intensities of the red light, the green light, and the blue light satisfy the relationship illustrated in FIG. 10C. Alternatively, the peak intensity of the green light and the peak intensity of the blue light may be reduced by placing a filter whose spectral transmittance for the green light and the blue light is low before the light source 50.

Even in Example 1 of the configuration of the light source 50 described above, the peak intensity of each of the green light and the blue light may be made lower than the peak intensity of the red light by adjusting an amount of the fluorescent body included in the wavelength conversion element 52. Alternatively, even in Example 1 of the configuration of the light source 50 described above, the peak intensity of the green light and the peak intensity of the blue light may be reduced by placing a filter whose spectral transmittance for the green light and the blue light is low before the light source 50.

Therefore, according to the measurement device 100B according to Embodiment 2, biological information of the living body 10 can be more accurately generated on the basis of the first signal and the second signal, as with the measurement device 100A according to Embodiment 1. Furthermore, according to the measurement device 100B according to Embodiment 2, biological information can be acquired without environmental light by irradiating the living body 10 with light emitted from the light source 50.

### Embodiment 3

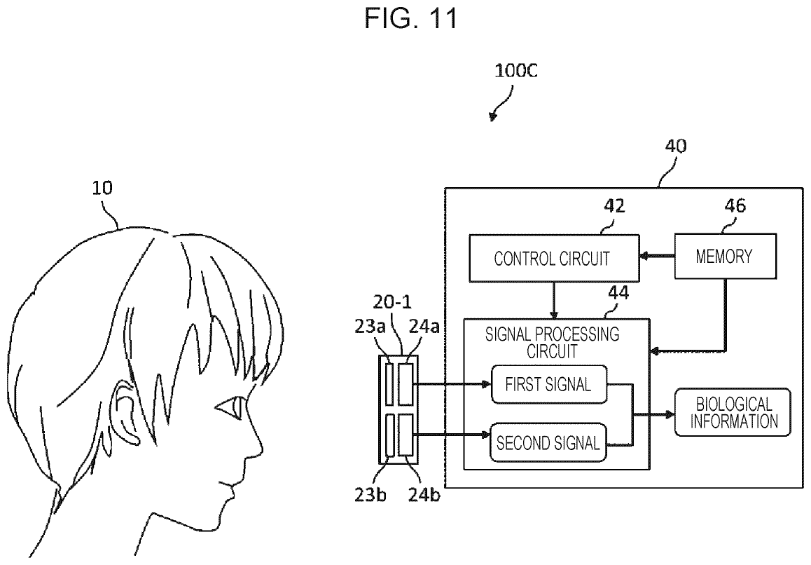

An example of a configuration of a measurement device according to Embodiment 3 of the present disclosure is described below with reference to FIG. 11. FIG. 11 schematically illustrates the configuration of the measurement device according to the exemplary Embodiment 3 of the present disclosure. A measurement device 100C illustrated in FIG. 11 does not include the bandpass filter 30 and includes an optical detection device 20-1 instead of the optical detection device 20 illustrated in FIG. 3, unlike the measurement device 100A illustrated in FIG. 3. The measurement device 100C may further include the light source 50 illustrated in FIG. 8.

The optical detection device 20-1 includes a first filter 23a configured to transmit red light and a second filter 23b configured to transmit one of blue light and green light. However, each of the first filter 23a and the second filter 23b may be, for example, an interference filter including a dielectric multi-layer instead of a typical color filter, unlike the first filter 22a and the second filter 22b illustrated in FIG. 3. The first filter 23a is configured to transmit red light, but suppresses transmission of at least a part of light in a wavelength range greater than or equal to 550 nm and less than or equal to 600 nm. It is therefore possible to suppress mixture of biological information in a first signal.

A configuration of the optical detection device 20-1 is described below in detail.

### Optical Detection Device 20-1

The optical detection device 20-1 includes at least one first filter 23a and at least one second filter 23b. The first filter 23a and the second filter 23b have been described above. The optical detection device 20-1 may further include at least one third filter configured to transmit the other one of the green light and the blue light. The third filter can be, for example, an interference filter including a dielectric multi-layer, as with the first filter 23a and the second filter 23b. The number of first filters 23a may be one or may be more than one. The same applies to the number of second filters 23b and the number of third filters. An example of arrangement of the first filter 23a, the second filter 23b, and the third filter has been described in the description of the measurement device 100A according to Embodiment 1.

The optical detection device 20-1 includes at least one first optical detection element 24a that detects light from the living body 10 through the first filter 23a and at least one second optical detection element 24b that detects light from the living body 10 through the second filter 23b. The optical detection device 20 may further include at least one third optical detection element that detects light from the living body 10 through the third filter. The first optical detection element 24a, the second optical detection element 24b, and the third optical detection element have been described in the description of the measurement device 100A according to Embodiment 1.

The first filter 22a, the second filter 22b, and the third filter, each of which is an interference filter including a dielectric multi-layer, can be, for example, directly laminated on the first optical detection element 24a, the second optical detection element 24b, and the third optical detection element, respectively. That is, the optical detection device 20-1 includes filters and optical detection elements that are integrally formed.

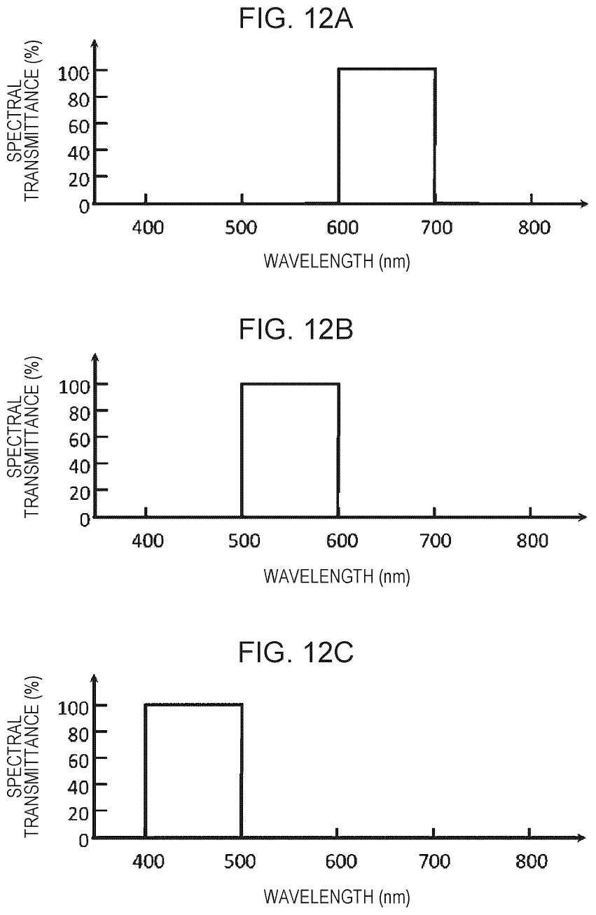

FIGS. 12A to 12C are graphs illustrating examples of spectral characteristics of the first filter 23a, the second filter 23b, and the third filter, respectively. In the examples illustrated in FIGS. 12A to 12C, the first filter 23a is a red filter, the second filter 23b is a green filter, and the third filter is a blue filter.

As illustrated in FIG. 12A, the first filter 23a has high spectral transmittance in a red wavelength range in a wavelength range of visible light, and has almost zero spectral transmittance in remaining wavelength ranges including a wavelength range greater than or equal to 550 nm and less than or equal to 600 nm. The high spectral transmittance can be, for example, greater than or equal to 60%, greater than or equal to 80%, greater than or equal to 90%, or greater than or equal to 95%. In the example illustrated in FIG. 12A, the red wavelength range is greater than or equal to 600 nm and less than or equal to 700 nm.

As illustrated in FIG. 12B, the second filter 23b has high spectral transmittance in a green wavelength range in the wavelength range of visible light, and has almost zero spectral transmittance in remaining wavelength ranges. In the example illustrated in FIG. 12B, the green wavelength range is greater than or equal to 500 nm and less than or equal to 600 nm.

As illustrated in FIG. 12C, the third filter has high spectral transmittance in a blue wavelength range in the wavelength range of visible light, and has almost zero spectral transmittance in remaining wavelength ranges. In the example illustrated in FIG. 12C, the blue wavelength range is greater than or equal to 400 nm and less than or equal to 500 nm.

Since each of the first filter 23a, the second filter 23b, and the third filter is an interference filter including a dielectric multi-layer, the spectral characteristics of the first filter 23a, the second filter 23b, and the third filter rapidly change. Therefore, the spectral characteristics of the first filter 23a hardly overlap the spectral characteristics of the second filter 23b and the spectral characteristics of the second filter. Similarly, the spectral characteristics of the second filter 23b hardly overlap the spectral characteristics of the third filter. Therefore, the first signal and the second signal hardly contain information of light in the same wavelength range. The same applies to the first signal and the third signal, and the same applies to the second signal and the third signal.

Therefore, according to the measurement device 100C according to Embodiment 3, biological information of the living body 10 can be effectively acquired on the basis of the first signal and the second signal as with the measurement device 100A according to Embodiment 1 without using the bandpass filter 30.

### Modifications

### Spectral Characteristics of Bandpass Filter 30

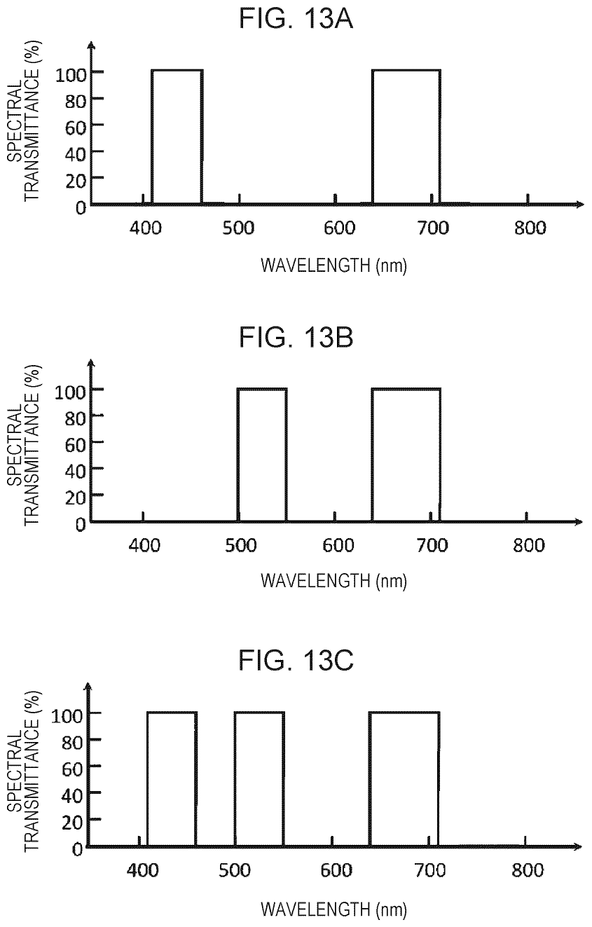

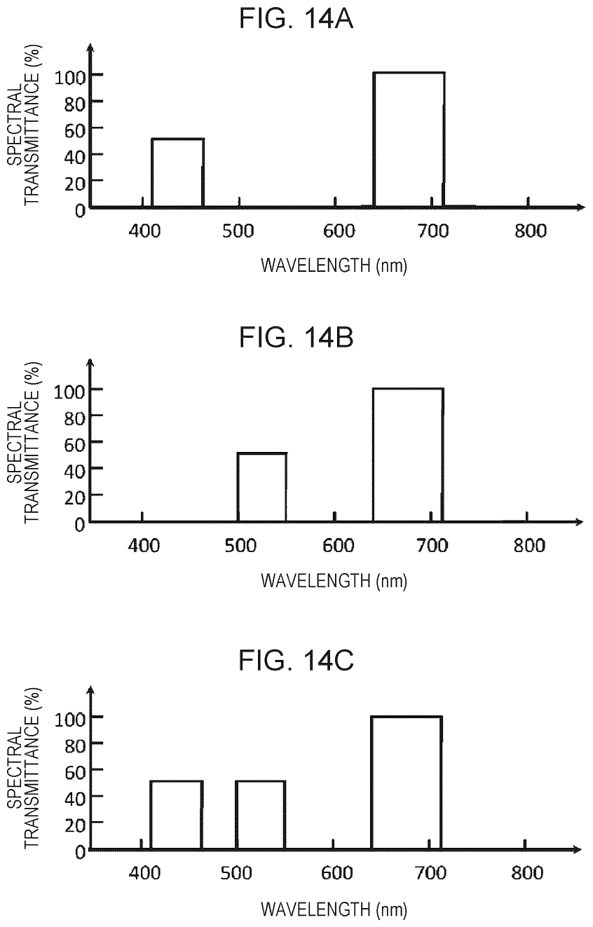

A modification of the bandpass filter 30 included in the measurement device 100A according to Embodiment 1 and the measurement device 100B according to Embodiment 2 is described below with reference to FIGS. 13A to 14C. The bandpass filter 30 included in the measurement device 100A and the measurement device 100B suppresses transmission of at least a part of light in the wavelength range greater than or equal to 550 nm and less than or equal to 600 nm in the wavelength range of visible light, and transmits light in remaining wavelength ranges. The bandpass filter 30 may suppress transmission of light in a part of the remaining wavelength ranges.

FIGS. 13A to 13C schematically illustrate other three examples of the spectral characteristics of the bandpass filter 30. In the example illustrated in FIG. 13A, the bandpass filter 30 has high spectral transmittance in the red wavelength range and has high spectral transmittance in the blue wavelength range in the wavelength range of visible light. Maximum spectral transmittance in the blue wavelength range is almost equal to maximum spectral transmittance in the red wavelength range. The bandpass filter 30 has almost zero spectral transmittance in the remaining wavelength ranges including the wavelength range greater than or equal to 550 nm and less than or equal to 600 nm. In the example illustrated in FIG. 13A, the red wavelength range is greater than or equal to 640 nm and less than or equal to 710 nm, and the blue wavelength range is greater than or equal to 410 nm and less than or equal to 460 nm. This bandpass filter 30 is advantageous in detecting red light to acquire the first signal and detecting blue light to acquire the second signal.

In the example illustrated in FIG. 13B, the bandpass filter 30 has high spectral transmittance in the red wavelength range and has high spectral transmittance in the green wavelength range in the wavelength range of visible light. Maximum spectral transmittance in the green wavelength range is almost equal to the maximum spectral transmittance in the red wavelength range. The bandpass filter 30 has almost zero spectral transmittance in remaining wavelength ranges including the wavelength range greater than or equal to 550 nm and less than or equal to 600 nm. In the example illustrated in FIG. 13B, the red wavelength range is greater than or equal to 640 nm and less than or equal to 710 nm, and the green wavelength range is greater than or equal to 500 nm and less than or equal to 550 nm. This bandpass filter 30 is advantageous in detecting red light to acquire the first signal and detecting green light to acquire the second signal.

In the example illustrated in FIG. 13C, the bandpass filter 30 has high spectral transmittance in the red wavelength range, has high spectral transmittance in the green wavelength range, and has high spectral transmittance in the blue wavelength range in the wavelength range of visible light. The maximum spectral transmittance in each of the green and blue wavelength ranges is almost equal to the maximum spectral transmittance in the red wavelength range. The bandpass filter 30 has almost zero spectral transmittance in remaining wavelength ranges including the wavelength range greater than or equal to 550 nm and less than or equal to 600 nm. In the example illustrated in FIG. 13C, the red wavelength range is greater than or equal to 640 nm and less than or equal to 710 nm, the green wavelength range is greater than or equal to 500 nm and less than or equal to 550 nm, and the blue wavelength range is greater than or equal to 410 nm and less than or equal to 460 nm. This bandpass filter 30 is advantageous in detecting red light to acquire the first signal, detecting one of green light and blue light to acquire the second signal, and detecting the other one of the green light and the blue light to acquire the third signal.

FIGS. 14A to 14C schematically illustrate still other three examples of the spectral characteristics of the bandpass filter 30. In the example illustrated in FIG. 14A, the maximum spectral transmittance in the blue wavelength range is lower than the maximum spectral transmittance in the red wavelength range, unlike the example illustrated in FIG. 13A. In the example illustrated in FIG. 14B, the maximum spectral transmittance in the green wavelength range is lower than the maximum spectral transmittance in the red wavelength range, unlike the example illustrated in FIG. 13B. In the example illustrated in FIG. 14C, the maximum spectral transmittance in each of the green and blue wavelength ranges is lower than the maximum spectral transmittance in the red wavelength range, unlike the example illustrated in FIG. 13C.

According to the relationship in spectral transmittance illustrated in FIGS. 14A to 14C, the intensity of the first signal can be increased by prolonging the exposure period, and a possibility that the intensity of the second signal and the intensity of the third signal are saturated can be reduced. In a case where the maximum spectral transmittance in each of the green and blue wavelength ranges is less than or equal to a half of the maximum spectral transmittance in the red wavelength range, the possibility can be effectively reduced.

Timing of Light Emission from Light Source 50

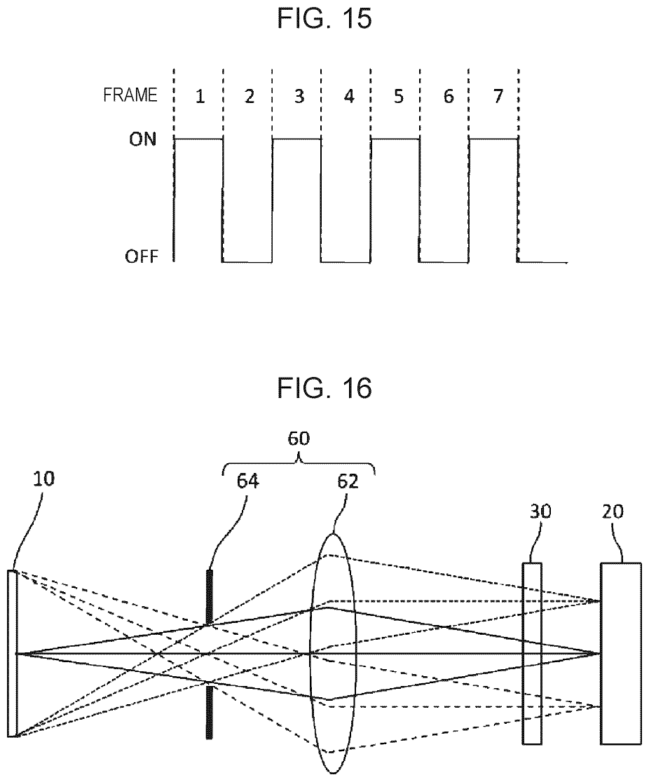

A modification of a timing of light emission from the light source 50 included in the measurement device 100B according to Embodiment 2 is described below with reference to FIG. 15. The light source 50 may intermittently emit light instead of constantly emitting light.

FIG. 15 schematically illustrates the modification of a timing of light emission from the light source 50. “ON” in FIG. 15 represents a state where light is being emitted from the light source 50, and “OFF” in FIG. 15 represents a state where emission of light from the light source 50 is stopped. In the example illustrated in FIG. 15, among sequentially numbered frames, light is emitted from the light source 50 in odd-numbered frames, and emission of light from the light source 50 is stopped in even-numbered frames. A frame rate can be, for example, 30 fps or 60 fps. In this way, light emission from the light source 50 and stoppage of light emission are switched every frame.

The optical detection device 20 detects reflected light generated by irradiating the living body 10 with environmental light in addition to light emitted from the light source 50 in an “ON” frame, and detects reflected light generated by irradiating the living body 10 with the environmental light without light emitted from the light source 50 in a next “OFF” frame. By subtracting a signal acquired in the next “OFF” frame from a signal acquired in the “ON” frame, a signal removing influence of the environmental light can be acquired.

### Use of Telecentric Lens

In a case where the bandpass filter 30 is an interference filter including a dielectric multi-layer, spectral characteristics of the bandpass filter 30 depend on an incident angle of incident light. To reduce the influence, the measurement device 100A according to Embodiment 1 and the measurement device 100B according to Embodiment 2 may further include a telecentric lens.

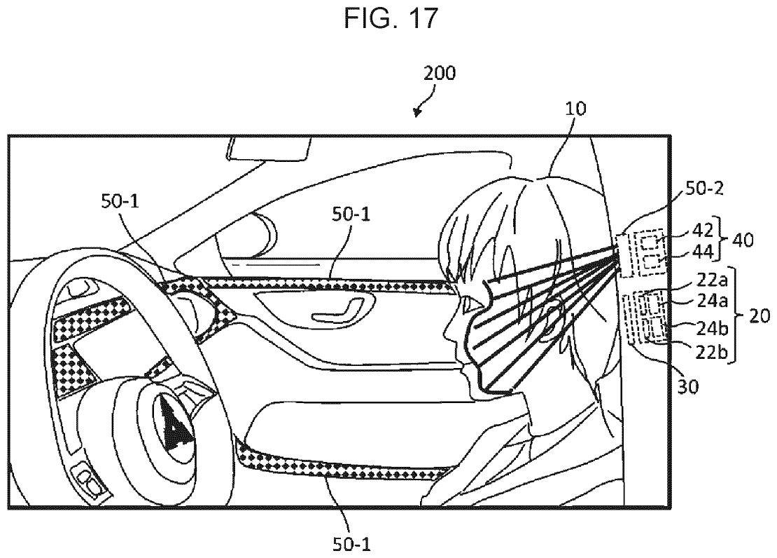

FIG. 16 schematically illustrates how light from the living body 10 that has passed through the telecentric lens 60 passes through the bandpass filter 30 and enters the optical detection device 20. As illustrated in FIG. 16, the telecentric lens 60 is placed before the bandpass filter 30. The telecentric lens 60 includes a convex lens 62 and a diaphragm 64 located at a focal point of the convex lens 62 on the living body 10 side. In FIG. 16, for simplification, the irradiated portion of the living body 10 is illustrated as a flat plate.

Among light from the living body 10, light that passes through the diaphragm 64 enters the convex lens 62, and light that does not pass through the diaphragm 64 does not enter the convex lens 62, and therefore a principal ray that passes through the convex lens 62 enters the bandpass filter 30 at the same incident angle irrespective of an angle of view. The same applies to an upper limit ray and a lower limit ray. Therefore, in a case where light from the living body 10 that has passed through the telecentric lens 60 is detected by the optical detection device 20 through the bandpass filter 30, the same signal intensity can be maintained even in a case where the angle of view changes due to movement of the living body 10.

In a case where the diaphragm 64 has a small f-number, sensitivity of light detection can be improved. In the telecentric lens 60, the diaphragm 64 having a small f-number can be used by combining convex lenses 62.

Note that the measurement device 100C according to Embodiment 3 does not include the bandpass filter 30, but includes the first filter 23a and the second filter 23b, each of which is an interference filter including a dielectric multi-layer. Therefore, for a reason similar to that described above, the measurement device 100C may further include the telecentric lens 60 before the optical detection device 20.

### Embodiment 4

An example of a configuration of a biometric measurement system according to Embodiment 4 in which a biometric measurement device is installed on a mobile object is described below with reference to FIG. 17. The biometric measurement system acquires biological information of an occupant of a mobile object. Although the mobile object is an automobile in the following description, the mobile object may be, for example, a ship or an airplane. Although the biometric measurement device is the measurement device 100A according to Embodiment 1 in the following description, the biometric measurement device may have, for example, a configuration obtained by removing the bandpass filter 30 from the measurement device 100A according to Embodiment 1.

### Biometric Measurement System

FIG. 17 schematically illustrates a configuration of a biometric measurement system according to an exemplary embodiment of the present disclosure. FIG. 17 also illustrates a living body 10 as an occupant of an automobile. Although the living body 10 is sitting on a driver's seat and is driving the mobile object in the example illustrated in FIG. 17, this example is not restrictive. The living body 10 may sit on a front passenger seat or a backseat. In the following description, the biometric measurement system is also referred to simply as a “measurement system”.

The measurement system 200 illustrated in FIG. 17 includes a light source 50-1 that is installed on the mobile object and emits light in a direction easily visible to the living body 10 and a light source 50-2 that is installed on the mobile object and emits light in a direction that is not easily visible to the living body 10. In the present specification, the light source 50-1 and the light source 50-2 are not distinguished in particular in some cases.

The measurement system 200 further includes an optical detection device 20 and a bandpass filter 30 that are installed on the mobile object. The optical detection device 20-1 illustrated in FIG. 11 may be used instead of the optical detection device 20 and the bandpass filter 30. The measurement system 200 further includes a processing device 40 installed on the mobile object. In the processing device 40, a control circuit 42 controls not only processing operation of a signal processing circuit 44, but also emission operation of the light source 50-1 and the light source 50-2.

Details of the light source 50-1, the light source 50-2, the optical detection device 20, the bandpass filter 30, and the processing device 40 are described below.

### Light Source 50-1 and Light Source 50-2

The light source 50-1 emits blue light for irradiating the living body 10. The living body 10 visually perceives the blue light. It is said that blue light has a strong relaxing effect, and exhibits a high calming effect particularly at a wavelength of 470 nm. Therefore, even in a case where the living body 10 who is driving is in a stressful condition or an excited condition, the blue light emitted from the light source 50-1 can calm the living body 10 down. The living body 10 can be guided to safe driving by creating a light environment centered on a blue color within an indoor space of the mobile object. The blue light emitted from the light source 50-1 is also useful for acquisition of the second signal.

The light source 50-2 emits blue light for irradiating the living body 10 separately from the light source 50-1. A cheek of the living body 10 is irradiated from an obliquely rearward direction with the blue light emitted from the light source 50-2. Since the living body 10 is irradiated not only with the blue light emitted from the light source 50-1, but also with the blue light emitted from the light source 50-2, the SN ratio of the second signal can be improved.

The light emitted from the light source 50-2 is not easily visible to the living body 10. Therefore, the light source 50-2 may emit red light, which is a hazard color. According to the configuration in which the light source 50-1 emits blue light and the light source 50-2 emits red light, a difference between the intensity of the first signal and the intensity of the second signal can be reduced to balance these intensities.

The light source 50-2 is not limited to a light source that emits blue light or red light and may be a light source that emits light of a different color. For example, the light source 50-2 may emit white light.

### Optical Detection Device 20 and Bandpass Filter 30

In the optical detection device 20, a first optical detection element 24a detects light from the living body 10 through a first filter 22a, more specifically, reflected light from the living body 10 originating from red light included in the light from the living body 10. In a case where the light source 50-1 and the light source 50-2 emit blue light, the red light can be, for example, light from interior lighting separately installed on the mobile object.

In the optical detection device 20, a second optical detection element 24b detects light from the living body 10 through a second filter 22b, more specifically, reflected light from the living body 10 originating from blue light included in the light from the living body 10. The bandpass filter 30 is placed before the optical detection device 20.

### Processing Device 40

In the processing device 40, the control circuit 42 causes the signal processing circuit 44 to generate biological information of the living body 10 on the basis of the first signal and the second signal. The control circuit 42 may further perform the following operation.

The control circuit 42 determines a psychological state of the living body 10 on the basis of the biological information of the living body 10. The psychological state of the living body 10 can be, for example, a stressful condition or an excited condition of the living body 10. The control circuit 42 causes the light source 50-1 to control output of the blue light on the basis of the psychological state of the living body 10. The control of output of the blue light is, for example, increasing or decreasing an intensity of the blue light.

More specific operation of the control circuit 42 is as follows. The control circuit 42 determines whether or not a level of stress or excitement of the living body 10 is high on the basis of the biological information of the living body 10. For example, in a case where a heart rate is greater than or equal to a predetermined threshold, the control circuit 42 determines that the level of stress or excitement of the living body 10 is high.

In a case where the control circuit 42 determines that the level of stress or excitement of the living body 10 is high, the control circuit 42 causes the light source 50-1 to increase the intensity of the blue light. This can calm the living body 10 down, thereby guiding the living body 10 to safe driving.

### Installation Positions of Constituent Elements

Installation positions of the constituent elements are as follows. By installing the constituent elements at appropriate positions by using the configuration of the mobile object, the biological information of the living body 10 can be effectively acquired.

### Light Source 50-1

In a case where the living body 10 is sitting on a driver's seat, the light source 50-1 can be, for example, installed on an interior side of a driver's door and/or near a steering wheel. In a case where the living body 10 is sitting on a front passage seat, the light source 50-1 can be, for example, installed on an interior side of a front passenger door and/or near a glove compartment. In a case where the living body 10 is sitting on a backseat, the light source 50-1 can be, for example, installed on an interior side of a rear door and/or on a rear side of a front seat. In this way, the light source 50-1 can be installed near a seat on which the living body 10 sits so that the blue light emitted from the light source 50-1 is visible to the living body 10.

### Light Source 50-2

In a case where the living body 10 is sitting on a driver's seat or on a front passenger seat, the light source 50-2 can be, for example, installed on a center pillar, as illustrated in FIG. 17. In a case where the living body 10 is sitting on a back seat, the light source 50-2 can be, for example, installed on a rear pillar. In either case, the following advantage is obtained.
The light source 50-2 does not obstruct a field of view of the living body 10.

### Optical Detection Device 20

In a case where the living body 10 is sitting on a driver's seat or on a front passenger seat, the optical detection device 20 can be, for example, installed on the center pillar of the automobile, as with the light source 50-2.

In this case, the following advantages are obtained.
The optical detection device 20 does not obstruct a field of view of the living body 10.
Since a distance from the living body 10 to the optical detection device 20 is short, the optical detection device 20 can effectively detect light from the living body 10.

In a case where the living body 10 is sitting on a driver's seat, the optical detection device 20 may be, for example, installed behind a center of the steering wheel of the automobile instead of the center pillar. In this case, the following advantages are obtained.
The optical detection device 20 is in front of the living body 10, and therefore light from the whole face of the living body 10 can be detected, and biological information can be stably measured.
The optical detection device 20 does not obstruct a field of view of the living body 10.
A monitoring camera for the living body 10 installed at the center of the steering wheel can also be used as the optical detection device 20.

In a case where the living body 10 is sitting on a front passenger seat, the optical detection device 20 may be, for example, installed on the glove compartment before the front passenger seat instead of the center pillar. In this case, the following advantages are obtained.
The optical detection device 20 is in front of the living body 10, and therefore light from the whole face of the living body 10 can be detected, and biological information can be stably measured.
The optical detection device 20 does not obstruct a field of view of the living body 10.

In a case where the living body 10 is sitting on a backseat, the optical detection device 20 can be, for example, installed on the rear pillar, as with the light source 50-2. In this case, the following advantages are obtained.
The optical detection device 20 does not obstruct a field of view of the living body 10.
Since a distance from the living body 10 to the optical detection device 20 is short, the optical detection device 20 can effectively detect light from the living body 10.

Note that since the bandpass filter 30 is placed before the optical detection device 20, the bandpass filter 30 is installed at a similar position to the optical detection device 20.

### Processing Device 40

The processing device 40 may be, for example, installed on the center pillar or may be installed at any position of the automobile, as with the light source 50-2. Alternatively, the processing device 40 may be installed at a place remote from the automobile. In a case where the processing device 40 is installed at a remote place, the processing device 40 wirelessly controls emission operation of the light source 50-1 and the light source 50-2 and wirelessly acquires a signal output from the optical detection device 20.

Therefore, according to the measurement system 200 according to Embodiment 4, biological information of the living body 10 who is an occupant of the mobile object can be effectively acquired by installing the constituent elements at appropriate positions by using the configuration of the mobile object. In this respect, even in a case where the first filter 22a included in the optical detection device 20 is a typical red filter, the measurement system 200 according to Embodiment 4 need not necessarily include the bandpass filter 30.

Furthermore, according to the measurement system 200 according to Embodiment 4, in a case where it is determined that the level of stress or excitement of the living body 10 is high, the living body 10 can be calmed down by increasing the intensity of the blue light emitted from the light source 50-1.

Note that although the light source 50-1 emits blue light in the measurement system according to Embodiment 4, the light source 50-1 may emit light of a color different from blue.

### Appendix

The following techniques are disclosed by the description of the above embodiments.

### Technique 1

A biometric measurement device including:
a bandpass filter;
an optical detection device; and
a processing circuit, wherein
the optical detection device includes a first filter configured to transmit red light, a second filter configured to transmit one of green light and blue light, a first optical detection element that detects light from a living body through the first filter, and a second optical detection element that detects light from the living body through the second filter,
the processing circuit generates biological information of the living body on the basis of first light detected by the first optical detection element and second light detected by the second optical detection element, and
the bandpass filter suppresses transmission of at least a part of light in a wavelength range greater than or equal to 550 nm and less than or equal to 600 nm that is included in light from the living body incident on the first optical detection element.

According to the biometric measurement device, mixture of the biological information in the first light can be suppressed by the bandpass filter, and it is therefore possible to effectively acquire the biological information on the basis of the first light and the second light.

### Technique 2

The biometric measurement device according to technique 1, in which

spectral characteristics of the first filter have a peak in a wavelength range greater than or equal to 550 nm and less than or equal to 800 nm.

According to the biometric measurement device, a typical red filter can be used as the first filter.

### Technique 3

The biometric measurement device according to technique 1 or 2, in which

spectral characteristics of the first filter have spectral transmittance greater than or equal to 30% in at least a part of the wavelength range greater than or equal to 550 nm and less than or equal to 600 nm.

According to the biometric measurement device, a typical red filter can be used as the first filter.

### Technique 4

The biometric measurement device according to any one of techniques 1 to 3, in which
the first optical detection element outputs a first signal based on the first light,
the second optical detection element outputs a second signal based on the second light, and
the processing circuit generates the biological information on the basis of a difference between a temporal change of an intensity of the first signal and a temporal change of an intensity of the second signal.

According to the biometric measurement device, the biological information can be generated by simple arithmetic processing, and therefore a computational load on the processing circuit can be lessened.

### Technique 5

The biometric measurement device according to any one of techniques 1 to 4, in which
the optical detection device further includes a third filter configured to transmit the other one of the green light and the blue light and a third optical detection element that detects light from the living body through the third filter, and
the processing circuit generates hue information of the living body on the basis of third light detected by the third optical detection element in addition to the first light and the second light.

According to the biometric measurement device, the hue information of the living body can be more accurately generated.

### Technique 6

The biometric measurement device according to technique 5, in which
the optical detection device includes first filters including the first filter, second filters including the second filter, third filters including the third filter, first optical detection elements including the first optical detection element, second optical detection elements including the second optical detection element, and third optical detection elements including the third optical detection element, and
the processing circuit generates the hue information on the basis of light detected by the first optical detection elements, light detected by the second optical detection elements, and light detected by the third optical detection elements, and determines a first optical detection element, a second optical detection element, and a third optical detection element that correspond to a position of a face of the living body among the first optical detection elements, the second optical detection elements, and the third optical detection elements on the basis of the hue information.

According to the biometric measurement device, the position of the face of the living body can be specified even in a case where the living body moves.

### Technique 7

The biometric measurement device according to any one of techniques 1 to 6, in which
maximum spectral transmittance of the bandpass filter in one of green and blue wavelength ranges is lower than maximum spectral transmittance of the bandpass filter in a red wavelength range.

According to the biometric measurement device, the intensity of the first signal can be increased by prolonging an exposure period, and a possibility that the intensity of the second signal is saturated can be reduced.

### Technique 8

The biometric measurement device according to any one of techniques 1 to 7, further including a light source that emits the red light and the one of the green light and the blue light for irradiating the living body.

According to the biometric measurement device, the biological information can be acquired without environmental light by using the light source.

### Technique 9

The biometric measurement device according to technique 8, in which
a peak intensity of the one of the green light and the blue light is lower than a peak intensity of the red light.

According to the biometric measurement device, the intensity of the first signal can be increased by prolonging an exposure period, and a possibility that the intensity of the second signal is saturated can be reduced.

### Technique 10

A biometric measurement device including:
an optical detection device; and
a processing circuit, wherein
the optical detection device includes a first filter configured to transmit red light, a second filter configured to transmit one of green light and blue light, a first optical detection element that detects light from a living body through the first filter, and a second optical detection element that detects light from the living body through the second filter,
the processing circuit generates biological information of the living body on the basis of first light detected by the first optical detection element and second light detected by the second optical detection element, and
the first filter is an interference filter including a dielectric multi-layer and suppresses transmission of at least a part of light in a wavelength range greater than or equal to 550 nm and less than or equal to 600 nm.

According to the biometric measurement device, mixture of the biological information in the first light can be suppressed by the first filter, and therefore the biological information can be effectively acquired on the basis of the first light and the second light.

### Technique 11

A biometric measurement system including:
a light source that is installed on a mobile object and emits blue light for irradiating an occupant of the mobile object;
an optical detection device that is installed on the mobile object and detects reflected light from the occupant generated by the blue light; and
a processing circuit, wherein
the optical detection device includes a first filter configured to transmit red light, a second filter configured to transmit the blue light, a first optical detection element that detects light from the occupant through the first filter, and a second optical detection element that detects the reflected light from the occupant through the second filter, and
the processing circuit generates biological information of the occupant on the basis of light detected by the first optical detection element and light detected by the second optical detection element.

According to the biometric measurement device, the biological information can be effectively acquired by installing the constituent elements at appropriate positions by using the configuration of the mobile object.

### Technique 12

The biometric measurement system according to technique 11, in which
the blue light includes light having a wavelength of 470 nm.

According to the biometric measurement device, the occupant can be calmed down by using light having a wavelength of 470 nm, which has a high calming effect.

### Technique 13

The biometric measurement system according to technique 11 or 12, in which
the processing circuit determines a psychological state of the occupant on the basis of the biological information and causes the light source to control output of the blue light on the basis of the psychological state.

According to the biometric measurement device, the occupant can be calmed down on the basis of the psychological state of the occupant.

### Technique 14

The biometric measurement system according to technique 13, in which
the processing circuit determines whether or not a level of stress or excitement of the occupant is high on the basis of the biological information, and
in a case where the processing circuit determines that the level of stress or excitement is high, the processing circuit causes the light source to increase an intensity of the blue light.

According to the biometric measurement device, the occupant can be calmed down in a case where the level of stress or excitement of the occupant is high.

### Technique 15

The biometric measurement system according to any one of techniques 11 to 14, in which
the light source is installed near a seat on which the occupant sits.

According to the biometric measurement device, the occupant can visually perceive the blue light.

### Technique 16

The biometric measurement system according to any one of techniques 11 to 15, in which
the mobile object is an automobile,
the light source and the optical detection device are installed on a center pillar or a rear pillar of the automobile, and
the optical detection device detects reflected light from a cheek of the occupant.

According to the biometric measurement device, the light source and the optical detection device do not obstruct the field of view of the occupant.

### Technique 17

The biometric measurement system according to any one of techniques 11 to 16, in which
the mobile object is an automobile, and
the optical detection device is installed on a steering wheel or a glove compartment of the automobile.

According to the biometric measurement device, the optical detection device is installed in front of the occupant, and therefore light from the whole face of the occupant can be detected, and the biological information can be stably measured.

### Technique 18

The biometric measurement device according to technique 10, further including a light source that emits the red light and the one of the green light and the blue light for irradiating the living body.

According to the biometric measurement device, the biological information can be acquired without environmental light by using the light source.

### Technique 19

The biometric measurement device according to technique 10 or 18, in which
the optical detection device further includes a third filter configured to transmit the other one of the green light and the blue light and a third optical detection element that detects light from the living body through the third filter, and
the processing circuit generates hue information of the living body on the basis of third light detected by the third optical detection element in addition to the first light and the second light.

According to the biometric measurement device, the hue information of the living body can be more accurately generated.

### Technique 20

The biometric measurement device according to technique 19, in which
the optical detection device includes first filters including the first filter, second filters including the second filter, third filters including the third filter, first optical detection elements including the first optical detection element, second optical detection elements including the second optical detection element, and third optical detection elements including the third optical detection element, and
the processing circuit generates the hue information on the basis of light detected by the first optical detection elements, light detected by the second optical detection elements, and light detected by the third optical detection elements, and determines a first optical detection element, a second optical detection element, and a third optical detection element that correspond to a position of a face of the living body among the first optical detection elements, the second optical detection elements, and the third optical detection elements on the basis of the hue information.

According to the biometric measurement device, the position of the face of the living body can be specified even in a case where the living body moves.

### Technique 21

The biometric measurement system according to any one of techniques 11 to 17, further including a bandpass filter that suppresses transmission of at least a part of light in the wavelength range greater than or equal to 550 nm and less than or equal to 600 nm that is included in the light from the occupant incident on the first filter.

According to the biometric measurement system, mixture of the biological information in the first light can be suppressed by the bandpass filter, and therefore the biological information can be effectively acquired on the basis of the first light and the second light.

### Technique 22

The biometric measurement system according to any one of techniques 11 to 17, in which
the first filter suppresses transmission of at least a part of light in the wavelength range greater than or equal to 550 nm and less than or equal to 600 nm.

According to the biometric measurement device, mixture of the biological information in the first light can be suppressed by the first filter, and therefore the biological information can be effectively acquired on the basis of the first light and the second light.

The technique of the present disclosure is useful, for example, for a biometric measurement device that acquires biological information. The technique of the present disclosure is also applicable, for example, to sensing for a living body, sensing for a medical purpose or a cosmetic purpose, and an in-vehicle sensing system.

What is claimed is:

1. A biometric measurement device comprising:
a bandpass filter;
an optical detection device; and
a processing circuit, wherein
the optical detection device includes a first filter configured to transmit red light, a second filter configured to transmit one of green light and blue light, a first optical detection element that detects light from a living body through the first filter, and a second optical detection element that detects light from the living body through the second filter,
the processing circuit generates biological information of the living body on a basis of first light detected by the first optical detection element and second light detected by the second optical detection element, and
the bandpass filter suppresses transmission of at least a part of light in a wavelength range greater than or equal to 550 nm and less than or equal to 600 nm that is included in light from the living body incident on the first optical detection element.

2. The biometric measurement device according to claim 1, wherein
spectral characteristics of the first filter have a peak in a wavelength range greater than or equal to 550 nm and less than or equal to 800 nm.

3. The biometric measurement device according to claim 1, wherein
spectral characteristics of the first filter have spectral transmittance greater than or equal to 30% in at least a part of the wavelength range greater than or equal to 550 nm and less than or equal to 600 nm.

4. The biometric measurement device according to claim 1, wherein
the first optical detection element outputs a first signal based on the first light,
the second optical detection element outputs a second signal based on the second light, and
the processing circuit generates the biological information on a basis of a difference between a temporal change of an intensity of the first signal and a temporal change of an intensity of the second signal.

5. The biometric measurement device according to claim 1, wherein
the optical detection device further includes a third filter configured to transmit an other one of the green light and the blue light and a third optical detection element that detects light from the living body through the third filter, and
the processing circuit generates hue information of the living body on a basis of third light detected by the third optical detection element in addition to the first light and the second light.

6. The biometric measurement device according to claim 5, wherein
the optical detection device includes first filters including the first filter, second filters including the second filter, third filters including the third filter, first optical detection elements including the first optical detection element, second optical detection elements including the second optical detection element, and third optical detection elements including the third optical detection element, and
the processing circuit generates the hue information on a basis of light detected by the first optical detection elements, light detected by the second optical detection elements, and light detected by the third optical detection elements, and determines a first optical detection element, a second optical detection element, and a third optical detection element that correspond to a position of a face of the living body among the first optical detection elements, the second optical detection elements, and the third optical detection elements on a basis of the hue information.

7. The biometric measurement device according to claim 1, wherein
maximum spectral transmittance of the bandpass filter in one of green and blue wavelength ranges is lower than maximum spectral transmittance of the bandpass filter in a red wavelength range.

8. The biometric measurement device according to claim 1, further comprising a light source that emits the red light and the one of the green light and the blue light for irradiating the living body.

9. The biometric measurement device according to claim 8, wherein
a peak intensity of the one of the green light and the blue light is lower than a peak intensity of the red light.

10. A biometric measurement device comprising:
an optical detection device; and
a processing circuit, wherein
the optical detection device includes a first filter configured to transmit red light, a second filter configured to transmit one of green light and blue light, a first optical detection element that detects light from a living body through the first filter, and a second optical detection element that detects light from the living body through the second filter,
the processing circuit generates biological information of the living body on a basis of first light detected by the first optical detection element and second light detected by the second optical detection element, and
the first filter is an interference filter including a dielectric multi-layer and suppresses transmission of at least a part of light in a wavelength range greater than or equal to 550 nm and less than or equal to 600 nm.

11. A biometric measurement system comprising:
a light source that is installed on a mobile object and emits blue light for irradiating an occupant of the mobile object;
an optical detection device that is installed on the mobile object and detects reflected light from the occupant generated by the blue light; and
a processing circuit, wherein
the optical detection device includes a first filter configured to transmit red light, a second filter configured to transmit the blue light, a first optical detection element that detects light from the occupant through the first filter, and a second optical detection element that detects the reflected light from the occupant through the second filter, and
the processing circuit generates biological information of the occupant on a basis of light detected by the first optical detection element and light detected by the second optical detection element.

12. The biometric measurement system according to claim 11, wherein
the blue light includes light having a wavelength of 470 nm.

13. The biometric measurement system according to claim 11, wherein
the processing circuit determines a psychological state of the occupant on a basis of the biological information and causes the light source to control output of the blue light on a basis of the psychological state.

14. The biometric measurement system according to claim 13, wherein
the processing circuit determines whether or not a level of stress or excitement of the occupant is high on a basis of the biological information, and
in a case where the processing circuit determines that the level of stress or excitement is high, the processing circuit causes the light source to increase an intensity of the blue light.

15. The biometric measurement system according to claim 11, wherein
the light source is installed near a seat on which the occupant sits.

16. The biometric measurement system according to claim 11, wherein
the mobile object is an automobile,
the light source and the optical detection device are installed on a center pillar or a rear pillar of the automobile, and
the optical detection device detects reflected light from a cheek of the occupant.

17. The biometric measurement system according to claim 11, wherein
the mobile object is an automobile, and
the optical detection device is installed on a steering wheel or a glove compartment of the automobile.
